SEQUENCE LISTING (2 sequences)

SEQ ID NO: 1 - protein, 1457 aa, Artificial Sequence
  source: /note=“Description of Artificial Sequence Synthetic polypeptide”
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - DNA, 5132 bp, Artificial Sequence
  source: /note=“Description of Artificial Sequence Synthetic polynucleotide”
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaagcttggaaccattgccaccttcagggggaggctgctggtgaatattaaccaagatcaccccagttaccggaggagcaaacagggactaagttcacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaagaggtaagggtttaagttatcgttagttcgtgcaccattaatgtttaattacctggagcacctgcctgaaatcatttttttttcaggttggctagtatgcagatcgagctctccacctgcttctttctgtgcctgttgagattctgcttcagcgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatccacccgtccttaagcgccatcagcgcgagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcatctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaggatccaataaaatatctttattttcattacatctgtgtgttggttttttgtgtgttttcctgtaacgatcgggctcgagcgcaggaacccctagtgatggagttggccactccctctctgcgcgctcgctcgctcactgaggccgcccgggctttgcccgggcggcctcagtgagcgagcgagcgcgcag

(19) United States
(12) Patent Application Publication
(10) Pub. No.: US 2024/0066146 A1
(43) Pub. Date: Feb. 29, 2024

(54) IMPROVED PHARMACEUTICAL COMPOSITIONS CONTAINING ADENO-ASSOCIATED VIRAL VECTOR

(71) Applicants: Sangamo Therapeutics, Inc., Brisbane, CA (US); Pfizer Inc., New York, NY (US)

(72) Inventors: Jessica Eileen Conner, St. Louis, MO (US); Lindsey Anne Crawford, Londonderry, NH (US); Robert Damitz, Chapel Hill, NC (US); Brendan Michael Davis, Somerville, MA (US); Cody Michael Hodge, St. Charles, MO (US); Michael Leland Kimmel II, St. Louis, MO (US); Tihami Qureshi Willard, St. Louis, MO (US); Phillip Ramsey, Daly City, CA (US); Daniel Joseph Thorne, St. Louis, MO (US); Anthony Lee Young, Edwardsville, IL (US)

(73) Assignees: Sangamo Therapeutics, Inc., Brisbane, CA (US); Pfizer Inc., New York, NY (US)

(21) Appl. No.: 18/258,027

(22) PCT Filed: Dec. 17, 2021
(86) PCT No.: PCT/US21/64222

§ 371 Date: Jun. 16, 2023

Related U.S. Application Data
(60) Provisional application No. 63/127,826, filed on Dec. 18, 2020.

(51) Int. Cl. A61K 48/00; A61K 38/37; A61K 47/02; A61K 47/10; A61K 47/26; C12N 15/86

(52) U.S. Cl. CPC A61K 48/005; A61K 38/37; A61K 47/02; A61K 47/10; A61K 47/26; C12N 15/86; C12N 2750/14143

(57) ABSTRACT

The present invention provides compositions comprising a recombinant AAV and one or more pharmaceutically acceptable excipients. The compositions have improved stability and shelf life as compared to other AAV compositions.

### CROSS-REFERENCE TO RELATED APPLICATION

This application claims priority from U.S. Application No. 63/127,826, filed Dec. 18, 2020, the content of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 1, 2021, is named 025297_WO029_SL.txt and is 19,967 bytes in size.

### BACKGROUND OF THE INVENTION

Gene therapy is a promising approach to treat genetic diseases. It introduces a healthy copy of the defective gene to the patient, or inactivates a mutated gene that is functioning improperly. A genetic disease that is of particular interest for gene therapy research is hemophilia A. Hemophilia A, also called classic hemophilia, is a X-linked genetic disease in which the blood clotting process is impaired due to a missing or defective gene encoding factor VIII. Patients with hemophilia A can bleed internally (e.g., into joints and muscles) or externally (e.g., due to minor cuts, trauma, or dental procedures). Normal plasma levels of factor VIII range from 50% to 100%. Mild hemophilia A is characterized by a level of 6% to 49% of factor VIII in the blood; patients typically bleed only after serious injury, trauma, or surgery. Moderate hemophilia A is characterized by a level of 1% to 5% of factor VIII in the blood; patients have bleeding episodes after injuries. Severe hemophilia A is characterized by a level of less than 1% of factor VIII in the blood; patients experience bleeding after injuries and also have frequent spontaneous bleeding episodes, often in their joints and muscles. See also the website of the National Hemophilia Foundation. Hemophilia A is commonly treated on-demand (upon bleeding) or prophylactically with replacement factor VIII. Some patients develop alloantibodies (aka inhibitors) against the replacement factor, rendering the therapy ineffective. Current therapies are burdensome because they require frequent intravenous injections and also are limited by resources in developing countries. Thus, gene therapy offers a promising approach to treating hemophilia A.

Recombinant adeno-associated virus (rAAV) has been explored as a platform for gene delivery in gene therapy. Adeno-associated virus (AAV) is a small non-enveloped virus belonging to the family Parvoviridae and the genus Dependoparvovirus. The virus is composed of a single-stranded DNA genome packaged into capsids assembled from three capsid proteins—viral protein (VP) 1, VP2, and VP3. Formulating rAAV preparations into pharmaceutical compositions for clinical use has been a challenge. Commonly used rAAV formulations have been observed to form visible precipitates over time or during lab-simulated stress tests. Left unaddressed, these precipitates represent a risk to patient safety. Physical stability issues observed with some rAAV formulations also could negatively impact the efficacy of products over the shelf life required for storage, shipment, and administration to patients (see, e.g., Wright et al., Molecular Therapy (2005) 12(1):171-8; Croyle et al., Gene Therapy (2001) 8:1281-90). Thus, there is a need for developing improved formulations for rAAV vectors so that their therapeutic potential in gene therapy can be fully implemented.

### SUMMARY OF THE INVENTION

The present disclosure provides stable rAAV vector formulations suitable for clinical administration. In an aspect, the disclosure provides a pharmaceutical composition comprising an rAAV vector, sodium chloride (NaCl), potassium chloride (KCl), disodium phosphate (Na₂HPO₄), monopotassium phosphate (KH₂PO₄), magnesium chloride (MgCl₂), a polyol (e.g., sucrose), and a poloxamer (e.g., poloxamer 188), optionally wherein the composition comprises no more than about 0.1 mM calcium chloride and has a pH of about 7.1 to about 7.5.

In some embodiments, the composition contains about 0.1 to about 2.0 mM (e.g., about 0.5 mM or higher, about 1.3 mM or higher, or about 1.4 mM) magnesium chloride.

In some embodiments, the composition contains about 150 to about 200 mM, optionally about 172 mM, sodium chloride.

In some embodiments, the composition contains about 2.5 to about 3.0 mM, optionally about 2.7 mM, potassium chloride.

In some embodiments, the composition contains about 5 to about 10 mM, optionally about 8 mM, disodium phosphate.

In some embodiments, the composition contains about 1.0 to about 2.0 mM, optionally about 1.5 mM, monopotassium phosphate.

In some embodiments, the composition contains about 0.5% to about 2% (w/v), optionally about 1% (w/v), sucrose.

In some embodiments, the composition contains about 0.01% to about 0.1% (w/v), optionally about 0.05% (w/v), poloxamer 188.

In particular embodiments, the present disclosure provides a pharmaceutical composition comprising an rAAV vector, about 171.81 mM sodium chloride, about 2.68 mM potassium chloride, about 8.10 mM disodium phosphate, about 1.47 mM monopotassium phosphate, about 1.40 mM magnesium chloride, about 1.00% (w/v) sucrose, and about 0.05% (w/v) poloxamer 188, optionally wherein the composition comprises no more than about 0.1 mM calcium chloride and has a pH of about 7.1 to about 7.5.

In other particular embodiments, the present disclosure provides a pharmaceutical composition comprising an rAAV vector, about 172 mM sodium chloride, about 2.68 mM potassium chloride, about 8.10 mM disodium phosphate, about 1.47 mM monopotassium phosphate, about 0.49 mM magnesium chloride, about 1.00% (w/v) sucrose, and about 0.05% (w/v) poloxamer 188, optionally wherein the composition comprises no more than about 0.1 mM calcium chloride and has a pH of about 7.1 to about 7.5.

In some embodiments, the rAAV in the present compositions comprises a genome comprising an expression cassette for a therapeutic protein, such as a human factor VIII polypeptide (e.g., SEQ ID NO:1). In particular embodiments, the AAV genome comprises SEQ ID NO:2 or nucleotides 131-5,024 of SEQ ID NO:2.

In some embodiments, the composition contains the rAAV at about 1.0E+12 to about 1.0E+14 vector genomes (vg) per mL, optionally about 1.0E+13 to about 5.0E+13 vg per mL (e.g., about 1.0E+13 vg per mL).

In some embodiments, the rAAV comprises an AAV6 capsid protein (e.g., having an AAV6 capsid). In some embodiments, the AAV genome comprises inverted terminal repeats (ITR) from AAV2.

In another aspect, the present disclosure provides a vial comprising 5-10 mL, optionally 6.4 mL, of the present composition. The vial may be made of, e.g., cyclo-olefin copolymer, and/or may have an in-place thermoplastic elastomer stopper.

In another aspect, the present disclosure provides a method of treating a patient in need of a therapeutic protein, comprising administering to the patient the present composition. In some embodiments, the present disclosure provides a method of increasing the serum level of factor VIII in a human subject in need thereof (e.g., a human subject having hemophilia A), comprising administering intravenously to the human subject the present composition where the rAAV encodes a human factor VIII polypeptide. Also provided are the pharmaceutical compositions for use in such treatment methods, and the use of the compositions for the manufacture of a medicament for use in such methods.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIGS. 1A-F summarize the characterization of the particulate matter found in samples of formulations containing SB-525.

FIG. 2 shows a schematic of buffer exchange by tangential flow filtration (TFF).

FIG. 3 shows the formulation, fill, and finish steps for the AAV6 vector formulations.

FIG. 4A shows the vector genome (vg) titer and infectious titer of AAV6 vector formulations following multiple freeze thaw cycles. Error bars displayed are either ±the percentage RSD from N≥3 sample measurements for particle concentration by MADLS, or ±the accepted test variability for vg titer, median tissue culture infectious dose (TCID₅₀), ELISA, and SE-LC. RSD: relative standard deviation. MADLS: multi-angle dynamic light scattering. SE-LC: size exclusion liquid chromatography.

FIG. 4B shows the capsid titer and particle concentration of AAV6 vector formulations following multiple freeze/thaw cycles. Error bars displayed are either ±the percentage RSD from N≥3 sample measurements for particle concentration by MADLS, or ±the accepted test variability for vg titer, TCID₅₀, ELISA, and SE-LC.

FIG. 4C is a table showing the general and purity quality attribute results of freeze/thaw AAV6 vector formulation samples.

FIG. 4D is a table showing the strength quality attribute results of freeze/thaw AAV6 vector formulation samples.

FIG. 5A shows vg and infectious titer over up to 6 months at ambient (25° C./60% RH) conditions. RH: relative humidity.

FIG. 5B shows capsid titer and particle concentration over up to 6 months at ambient (25° C./60% RH) conditions.

FIG. 5C is a table showing the general and purity quality attribute results of ambient (25° C./60% RH) incubated samples. ND: no data. NAA: no analysis available.

FIG. 5D is a table showing the strength quality attribute results of ambient (25° C./60% RH) incubated samples.

FIG. 6A shows vg and infectious titer over 3 months at stressed (40° C./75% RH) conditions.

FIG. 6B shows capsid titer and particle concentration over up to 7 months at stressed (40° C./75% RH) conditions.

FIG. 6C is a table showing the general and purity quality attribute results of stressed (40° C./75%RH) incubated samples.

FIG. 6D is a table showing the strength quality attribute results of stressed (40° C./75% RH) incubated samples.

FIG. 7 is a table showing quality attribute with associated and failure criteria for stability samples.

FIG. 8 is a table showing the inter-sample formulation assessment summary.

FIG. 9 is a table showing the study endpoint trendline and formulation assessments for general and purity quality attributes.

FIG. 10 is a table showing the long term (24 month) stability of the SB-525 drug product at the intended storage temperature (−70° C.).

### DETAILED DESCRIPTION OF THE INVENTION

The present disclosure provides pharmaceutical compositions comprising AAV vectors and one or more pharmaceutically acceptable excipients. The present AAV vector compositions may comprise rAAV whose genome carries an expression cassette for a protein of interest (e.g., a therapeutic protein). The present inventors have unexpectedly discovered that AAV vector formulations that are essentially calcium free (e.g., having no added calcium in the formulation) have improved stability and shelf life as compared to prior composition. Calcium is typically included in prior formulations due to the conventional belief that calcium would improve the stability of AAV compositions (see, e.g., Turnbull et al., Hum Gene Ther. (2000) 11(4):629-35; Cotmore et al., J Virol. (2010) 84(4):1945-56). The inventors have discovered that the present AAV vector compositions have improved appearance (e.g., clarity and lack of color), more stable pH, and less aggregates (as determined by product quality attributes under freeze/thaw cycles and accelerated stability conditions). Because the AAV vector product formulated without calcium has demonstrated better product stability, calcium ions are not required for product performance or stability.

### I. Preparation of Recombinant AAV

The viral preparations described herein may be obtained by any known production systems, such as mammalian cell AAV production systems (e.g., those based on 293T or HEK293 cells) and insect cell AAV production systems (e.g., those based on sf9 insect cells and/or those using baculoviral helper vectors). The viral preparations may be purified from the cell cultures by using well known techniques such as discontinuous cesium chloride density gradients (see, e.g., Grieger, Mol Ther Methods Clin Dev. (2016) 3:16002).

The present compositions may comprise AAV of any or a combination of a variety of AAV serotypes, such as AAV1, AAV2, AAV3, AAV3B, AAV4, AAVS, AAV6, AAV7, AAV8, AAV8.2, AAV9, AAVrh10, AAV10, and AAV11, as well as variants, hybrids, chimera or pseudo-types thereof. By “pseudo-typed” or “cross-packaged” rAAV is meant a recombinant AAV whose capsid is replaced with the capsid of another AAV serotype, to, for example, alter transduction efficacy or tropism profiles of the virus (see, e.g., Balaji et al., J Surg Res. (2013) 184(1):691-8). By “chimeric” or “hybrid” rAAV is meant a recombinant AAV whose capsid is assembled from capsid proteins derived from different serotypes and/or whose capsid proteins are chimeric proteins with sequences derived from different serotypes (e.g., serotypes 1 and 2; see, e.g., Hauck et al., Mol Ther. (2003) 7(3):419-25). For example, the present compositions may comprise recombinant AAV whose genome such as the ITRs is derived from one serotype such as AAV2 while the capsids are derived from another serotype; e.g., AAV2/8, AAV2/5, AAV2/6, AAV2/9, or AAV2/6/9. See, e.g., U.S. Pat. Nos. 7,198,951 and 9,585,971.

### II. Formulation of Recombinant AAV

Once purified, the AAV preparations can be formulated as described herein, for example, by buffer exchange through tangential flow filtration, normal flow filtration using stir-cells, gel filtration, dialysis, column chromatography, and/or desalting columns, to arrive at a composition comprising the desired ingredients. By way of illustration, the purified viral preparation may be concentrated first by ultrafiltration (UF) and then diafiltrated (DF) with 10 times or more equivalent volumes of the desired aqueous formulation solution. See also the Examples below.

The formulation solution may comprise tonicity agents, stabilizing agents, surfactants, and buffering agents. Buffering agents may include, for example, acetate, succinate (e.g., disodium succinate hexahydrate), succinic acid, gluconate, citrate, histidine, acetic acid, phosphate, phosphoric acid, ascorbate, ascorbic acid, tartaric acid, malate, maleic acid, glycine, lactate, lactic acid, bicarbonate, carbonic acid, sodium benzoate, benzoic acid, edetate, imidazole, tris, and mixtures thereof. In some embodiments, the formulation solution contains sodium chloride and/or potassium chloride at, e.g., about 150-200 mM (e.g., about 150 mM, about 155 mM, about 160 mM, about 165 mM, about 168 mM, about 170 mM, about 171 mM, about 171.1 mM, about 171.2 mM, about 171.3 mM , about 171.4 mM , about 171.5 mM , about 171.6 mM , about 171.7 mM , about 171.8 mM , about 171.9 mM , or about 172 mM) and about 2.5-3.0 mM (e.g., about 2.5 mM, about 2.6 mM, about 2.61 mM, about 2.61 mM, about 2.63 mM, about 2.64 mM, about 2.65 mM, about 2.66 mM, about 2.67 mM, about 2.68 mM, about 2.69 mM, or about 2.7 mM, about 2.8 mM, about 2.9 mM, about 3.0 mM), respectively.

The formulation solution may be phosphate-buffered, e.g., by disodium phosphate and/or monopotassium phosphate. In some embodiments, the total phosphate ion concentration in the formulation solution is about 8-12 mM (e.g., about 9.6 mM or about 9.57 mM). In certain embodiments, the formulation solution contains about 5-10 mM (e.g., about 5 mM, about 6 mM, about 7 mM, about 7.9 mM, about 8 mM or about 8.1 mM, about 8.2 mM, about 8.5 mM, about 9 mM, or about 10 mM) disodium phosphate and about 1-2 mM (e.g., about 1 mM, about 1.2 mM, about 1.3 mM, about 1.45 mM, about 1.47 mM, about 1.48 mM, about 1.49 mM, about 1.5, about 1.6 mM, about 1.7 mM, about 1.8 mM, about 1.9 Mm, or about 2 mM) monopotassium phosphate. The formulation may have a pH of about 6.5-8.0 (e.g., about 7.1-7.5, about 7.1, about 7.2, about 7.3, about 7.4, or about 7.5).

The formulation solution may contain magnesium but no added calcium. In some embodiments, the formulation solution contains about 0.1 to 2.0 mM (e.g., about 0.5 to about 1.4 mM, about 0.1 mM, about 0.2 mM, about 0.3 mM, about 0.4 mM, about 0.42 mM, about 0.44 mM, about 0.45 mM, about 0.46 mM, about 0.47 mM, about 0.48 mM, about 0.49 mM, about 0.5, about 0.55 mM, or about 0.6 mM,) magnesium chloride. Although the formulation solution contains no added calcium, the pharmaceutical composition made up from the AAV preparation and the formulation solution may have a trace amount of calcium that is carried over for the AAV manufacturing and purification process. For example, the pharmaceutical composition may contain no more than about 0.10 mM (e.g., no more than about 0.09, 0.07, 0.05, 0.03, or 0.01 mM) calcium when measured by a colorimetric assay. In some embodiments, the pharmaceutical composition contains no detectable calcium as measured by a colorimetric assay. In some embodiments, the pharmaceutical composition contains no calcium (i.e., 0 mM calcium).

The formulation solution may contain a polyol such as mannitol, trehalose, sorbitol, erythritol, isomalt, lactitol, maltitol, xylitol, glycerol, lactitol, ethylene glycol, propylene glycol, polyethylene glycol, inositol, fructose, glucose, mannose, sucrose, sorbose, xylose, lactose, maltose, dextran, pullulan, dextrin, cyclodextrins, soluble starch, hydroxyethyl starch, water-soluble glucans, or mixtures thereof. In some embodiments, the formulation solution contains about 0.5% to 2% (e.g., about 0.5%, about 0.6%, about 0.7%, about 0.8%, about 0.9%, or about 1%) (w/v) sucrose.

The formulation solution may contain a nonionic or ionic hydrophilic surfactant. Example of surfactants are a polysorbate, poloxamer, triton, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl- taurate, dihydroxypropyl PEG-5 linoleammonium chloride, polyethylene glycol, polypropylene glycol, sorbitan monoesters (e.g., Spans), acid esters of glycerol, and mixtures thereof. In some embodiments, the surfactant can be polysorbate (PS) 20, PS-21, PS-40, PS-60, PS-61, PS-65, PS-80, PS-81, PS-85, PEG-3350, poloxamer 188, and mixtures thereof. In some embodiments, the formulation solution contains about 0.01% to 0.1% (e.g., about 0.01%, about 0.02%, about 0.03%, about 0.04%, about 0.05%, about 0.06%, about 0.07%, about 0.08%, about 0.09%, or about 0.1%) (w/v) poloxamer 188.

In particular embodiments, the pharmaceutical composition comprises an AAV vector in Formulation F2 or F3. The ingredients for F2 and F3 are shown in Table A below. Dulbecco's phosphate buffered saline (DPBS) with calcium and magnesium is a commonly used formulation for cell culture applications. F0 is another prior, calcium-containing formulation. As shown in the Examples below, F2 and F3 are superior to DPBS and F0 in formulating AAV.

TABLE A
Formulation Comparison

| Component (unit) | DPBS | F0 | F2 | F3 |
|---|---|---|---|---|
| NaCl (mM) | 136.89 | 171.80 | 171.80 | 171.80 |
| KCl (mM) | 2.68 | 2.68 | 2.68 | 2.68 |
| Na₂HPO₄ (mM) | 8.10 | 8.10 | 8.10 | 8.10 |
| KH₂PO₄ (mM) | 1.47 | 1.47 | 1.47 | 1.47 |
| CaCl₂ (mM) | 0.90 | 0.90 | 0.0 | 0.0 |
| MgCl₂ (mM) | 0.50 | 0.50 | 0.49 | 1.40 |
| Sucrose (% w/v) | 0.0 | 1.00% | 1.00% | 1.00% |
| Poloxamer 188 (% w/v) | 0.0 | 0.05% | 0.05% | 0.05% |

As used herein, the concentrations of the various ingredients in the formulations may be expressed with zero, one, or two decimal places. Thus, for example, in F2, the concentrations of NaCl, KCl, Na₂HPO₄, KH₂PO₄, MgCl₂, and sucrose may be expressed respectively as 171.80 (or 171.8 or 172) mM, 2.68 (or 2.7) mM, 8.10 (or 8.1 or 8) mM, 1.47 (or 1.5) mM, 0.49 (or 0.5) mM, and 1.00% (or 1.0% or 1%) (w/v). In F3, the concentrations of NaCl, KCl, Na₂HPO₄, KH₂PO₄, MgCl₂, and sucrose may be expressed respectively as 171.80 (or 171.8 or 172) mM, 2.68 (or 2.7) mM, 8.10 (or 8.1 or 8) mM, 1.47 (or 1.5) mM, 1.40 (or 1.4) mM, and 1.00% (or 1.0% or 1%) (w/v).

The pharmaceutical compositions may further comprise one or more preservatives such as ascorbic acid (vitamin C), sulfites, sorbates, benzoates, phenol, m-cresol, benzyl alcohol, benzalkonium chloride, phenoxyethanol, and/or parabens (e.g., methyl paraben). In some embodiments, the pharmaceutical compositions do not contain any added preservatives.

The pharmaceutical compositions may comprise also other reagents that enhance the effectiveness of the pharmaceutical composition. The pharmaceutical composition may contain delivery vehicles such as liposomes, nanocapsules, microparticles, microspheres, lipid particles, and vesicles.

### III. Exemplary Recombinant AAV

In exemplary embodiments, the present disclosure provides improved pharmaceutical compositions comprising rAAV vector for factor VIII gene therapy. The rAAV vector, termed PF-07055480/SB-525 (or “SB-525” herein), is of the 2/6 pseudo serotype, which comprises an AAV6 capsid and a recombinant genome having AAV2 inverted terminal repeats (ITR). SB-525's genome carries an expression cassette encoding a B-domain-deleted (BDD) version of human factor VIII (FVIII). See, e.g., WO 2017/074526 (sequence #37). SB-525 is administered as an intravenous (IV) dose and possesses liver-specific tropism to provide long-term hepatic production of factor VIII protein in patients with hemophilia A. See WO 2020/028830. The secreted FVIII protein has the same amino acid sequence as approved recombinant anti-hemophilic factors (Refacto® and Xyntha®)

SB-525's genome comprises an expression cassette for a human factor VIII BDD version, which has the amino acid sequence shown below:

(SEQ ID NO: 1)

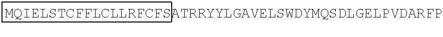

PRVPKSFPFNTSVVYKKTLFVEFTDHLFNIAKPRPPWMGLLGPTIQAEVY
DTVVITLKNMASHPVSLHAVGVSYWKASEGAEYDDQTSQREKEDDKVFPG
GSHTYVWQVLKENGPMASDPLCLTYSYLSHVDLVKDLNSGLIGALLVCRE
GSLAKEKTQTLHKFILLFAVEDEGKSWHSETKNSLMQDRDAASARAWPKM
HTVNGYVNRSLPGLIGCHRKSVYWHVIGMGTTPEVHSIFLEGHTFLVRNH
RQASLEISPITFLTAQTLLMDLGQFLLFCHISSHQHDGMEAYVKVDSCPE
EPQLRMKNNEEAEDYDDDLTDSEMDVVRFDDDNSPSFIQIRSVAKKHPKT
WVHYIAAEEEDWDYAPLVLAPDDRSYKSQYLNNGPQRIGRKYKKVRFMAY
TDETFKTREAIQHESGILGPLLYGEVGDTLLIIFKNQASRPYNIYPHGIT
DVRPLYSRRLPKGVKHLKDFPILPGEIFKYKWTVTVEDGPTKSDPRCLTR
YYSSFVNMERDLASGLIGPLLICYKESVDQRGNQIMSDKRNVILFSVFDE
NRSWYLTENIQRFLPNPAGVQLEDPEFQASNIMHSINGYVFDSLQLSVCL
HEVAYWYILSIGAQTDFLSVFFSGYTFKHKMVYEDTLTLFPFSGETVFMS
MENPGLWILGCHNSDFRNRGMTALLKVSSCDKNTGDYYEDSYEDISAYLL
SKNNAIEPRSFSQNPPVLKRHQREITRTTLQSDQEEIDYDDTISVEMKKE
DFDIYDEDENQSPRSFQKKTRHYFIAAVERLWDYGMSSSPHVLRNRAQSG
SVPQFKKVVFQEFTDGSFTQPLYRGELNEHLGLLGPYIRAEVEDNIMVTF
RNQASRPYSFYSSLISYEEDQRQGAEPRKNFVKPNETKTYFWKVQHHMAP
TKDEFDCKAWAYFSDVDLEKDVHSGLIGPLLVCHTNTLNPAHGRQVTVQE
FALFFTIFDETKSWYFTENMERNCRAPCNIQMEDPTFKENYRFHAINGYI
MDTLPGLVMAQDQRIRWYLLSMGSNENIHSIHFSGHVFTVRKKEEYKMAL
YNLYPGVFETVEMLPSKAGIWRVECLIGEHLHAGMSTLFLVYSNKCQTPL
GMASGHIRDFQITASGQYGQWAPKLARLHYSGSINAWSTKEPFSWIKVDL
LAPMIIHGIKTQGARQKFSSLYISQFIIMYSLDGKKWQTYRGNSTGTLMV
FFGNVDSSGIKHNIFNPPIIARYIRLHPTHYSIRSTLRMELMGCDLNSCS
MPLGMESKAISDAQITASSYFTNMFATWSPSKARLHLQGRSNAWRPQVNN
PKEWLQVDFQKTMKVTGVTTQGVKSLLTSMYVKEFLISSSQDGHQWTLFF
QNGKVKVFQGNQDSFTPVVNSLDPPLLTRYLRIHPQSWVHQIALRMEVLG
CEAQDLY

The signal peptide portion of SEQ ID NO:1 is shown in box above and is cleaved off when the protein is secreted. The SB-525 genome comprises the following nucleotide sequence:

| (SEQ ID NO:2) | |
|---|---|
| 1 | 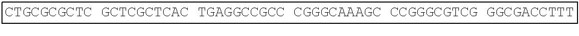 |
| 61 | 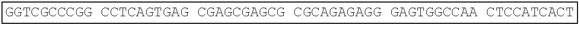 |
| 121 | 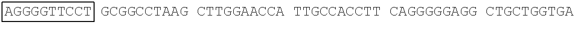 |
| 181 | ATATTAACCA AGATCACCCC AGTTACCGGA GGAGCAAACA GGGACTAAGT TCACACGCGT |
| 241 | GGTACCGTCT GTCTGCACAT TTCGTAGAGC GAGTGTTCCG ATACTCTAAT CTCCCTAGGC |
| 301 | AAGGTTCATA TTTGTGTAGG TTACTTATTC TCCTTTTGTT GACTAAGTCA ATAATCAGAA |
| 361 | TCAGCAGGTT TGGAGTCAGC TTGGCAGGGA TCAGCAGCCT GGGTTGGAAG GAGGGGGTAT |
| 421 | AAAAGCCCCT TCACCAGGAG AAGCCGTCAC ACAGATCCAC AAGCTCCTGA AGAGGTAAGG |
| 481 | GTTTAAGTTA TCGTTAGTTC GTGCACCATT AATGTTTAAT TACCTGGAGC ACCTGCCTGA |
| 541 | AATCATTTTT TTTTCAGGTT GGCTAGTATG CAGATCGAGC TCTCCACCTG CTTCTTTCTG |
| 601 | TGCCTGTTGA GATTCTGCTT CAGCGCCACC AGGAGATACT ACCTGGGGGC TGTGGAGCTG |
| 661 | AGCTGGGACT ACATGCAGTC TGACCTGGGG GAGCTGCCTG TGGATGCCAG GTTCCCCCCC |
| 721 | AGAGTGCCCA AGAGCTTCCC CTTCAACACC TCTGTGGTGT ACAAGAAGAC CCTGTTTGTG |
| 781 | GAGTTCACTG ACCACCTGTT CAACATTGCC AAGCCCAGGC CCCCCTGGAT GGGCCTGCTG |
| 841 | GGCCCCACCA TCCAGGCTGA GGTGTATGAC ACTGTGGTGA TCACCCTGAA GAACATGGCC |
| 901 | AGCCACCCTG TGAGCCTGCA TGCTGTGGGG GTGAGCTACT GGAAGGCCTC TGAGGGGGCT |
| 961 | GAGTATGATG ACCAGACCAG CCAGAGGGAG AAGGAGGATG ACAAGGTGTT CCCTGGGGGC |
| 1021 | AGCCACACCT ATGTGTGGCA GGTGCTGAAG GAGAATGGCC CCATGGCCTC TGACCCCCTG |
| 1081 | TGCCTGACCT ACAGCTACCT GAGCCATGTG GACCTGGTGA AGGACCTGAA CTCTGGCCTG |
| 1141 | ATTGGGGCCC TGCTGGTGTG CAGGGAGGGC AGCCTGGCCA AGGAGAAGAC CCAGACCCTG |
| 1201 | CACAAGTTCA TCCTGCTGTT TGCTGTGTTT GATGAGGGCA AGAGCTGGCA CTCTGAAACC |
| 1261 | AAGAACAGCC TGATGCAGGA CAGGGATGCT GCCTCTGCCA GGGCCTGGCC CAAGATGCAC |
| 1321 | ACTGTGAATG GCTATGTGAA CAGGAGCCTG CCTGGCCTGA TTGGCTGCCA CAGGAAGTCT |
| 1381 | GTGTACTGGC ATGTGATTGG CATGGGCACC ACCCCTGAGG TGCACAGCAT CTTCCTGGAG |
| 1441 | GGCCACACCT TCCTGGTCAG GAACCACAGG CAGGCCAGCC TGGAGATCAG CCCCATCACC |
| 1501 | TTCCTGACTG CCCAGACCCT GCTGATGGAC CTGGGCCAGT TCCTGCTGTT CTGCCACATC |
| 1561 | AGCAGCCACC AGCATGATGG CATGGAGGCC TATGTGAAGG TGGACAGCTG CCCTGAGGAG |
| 1621 | CCCCAGCTGA GGATGAAGAA CAATGAGGAG GCTGAGGACT ATGATGATGA CCTGACTGAC |
| 1681 | TCTGAGATGG ATGTGGTGAG GTTTGATGAT GACAACAGCC CCAGCTTCAT CCAGATCAGG |
| 1741 | TCTGTGGCCA AGAAGCACCC CAAGACCTGG GTGCACTACA TTGCTGCTGA GGAGGAGGAC |
| 1801 | TGGGACTATG CCCCCCTGGT GCTGGCCCCT GATGACAGGA GCTACAAGAG CCAGTACCTG |
| 1861 | AACAATGGCC CCCAGAGGAT TGGCAGGAAG TACAAGAAGG TCAGGTTCAT GGCCTACACT |
| 1921 | GATGAAACCT TCAAGACCAG GGAGGCCATC CAGCATGAGT CTGGCATCCT GGGCCCCCTG |
| 1981 | CTGTATGGGG AGGTGGGGGA CACCCTGCTG ATCATCTTCA AGAACCAGGC CAGCAGGCCC |
| 2041 | TACAACATCT ACCCCCATGG CATCACTGAT GTGAGGCCCC TGTACAGCAG GAGGCTGCCC |
| 2101 | AAGGGGGTGA AGCACCTGAA GGACTTCCCC ATCCTGCCTG GGGAGATCTT CAAGTACAAG |
| 2161 | TGGACTGTGA CTGTGGAGGA TGGCCCCACC AAGTCTGACC CCAGGTGCCT GACCAGATAC |
| 2221 | TACAGCAGCT TTGTGAACAT GGAGAGGGAC CTGGCCTCTG GCCTGATTGG CCCCCTGCTG |
| 2281 | ATCTGCTACA AGGAGTCTGT GGACCAGAGG GGCAACCAGA TCATGTCTGA CAAGAGGAAT |
| 2341 | GTGATCCTGT TCTCTGTGTT TGATGAGAAC AGGAGCTGGT ACCTGACTGA GAACATCCAG |
| 2401 | AGGTTCCTGC CCAACCCTGC TGGGGTGCAG CTGGAGGACC CTGAGTTCCA GGCCAGCAAC |
| 2461 | ATCATGCACA GCATCAATGG CTATGTGTTT GACAGCCTGC AGCTGTCTGT GTGCCTGCAT |
| 2521 | GAGGTGGCCT ACTGGTACAT CCTGAGCATT GGGGCCCAGA CTGACTTCCT GTCTGTGTTC |
| 2581 | TTCTCTGGCT ACACCTTCAA GCACAAGATG GTGTATGAGG ACACCCTGAC CCTGTTCCCC |
| 2641 | TTCTCTGGGG AGACTGTGTT CATGAGCATG GAGAACCCTG GCCTGTGGAT TCTGGGCTGC |
| 2701 | CACAACTCTG ACTTCAGGAA CAGGGGCATG ACTGCCCTGC TGAAAGTCTC CAGCTGTGAC |
| 2761 | AAGAACACTG GGGACTACTA TGAGGACAGC TATGAGGACA TCTCTGCCTA CCTGCTGAGC |
| 2821 | AAGAACAATG CCATTGAGCC CAGGAGCTTC AGCCAGAATC CACCCGTCCT TAAGCGCCAT |
| 2881 | CAGCGCGAGA TCACCAGGAC CACCCTGCAG TCTGACCAGG AGGAGATTGA CTATGATGAC |
| 2941 | ACCATCTCTG TGGAGATGAA GAAGGAGGAC TTTGACATCT ACGACGAGGA CGAGAACCAG |
| 3001 | AGCCCCAGGA GCTTCCAGAA GAAGACCAGG CACTACTTCA TTGCTGCTGT GGAGAGGCTG |
| 3061 | TGGGACTATG GCATGAGCAG CAGCCCCCAT GTGCTGAGGA ACAGGGCCCA GTCTGGCTCT |
| 3121 | GTGCCCCAGT TCAAGAAGGT GGTGTTCCAG GAGTTCACTG ATGGCAGCTT CACCCAGCCC |
| 3181 | CTGTACAGAG GGGAGCTGAA TGAGCACCTG GGCCTGCTGG GCCCCTACAT CAGGGCTGAG |
| 3241 | GTGGAGGACA ACATCATGGT GACCTTCAGG AACCAGGCCA GCAGGCCCTA CAGCTTCTAC |
| 3301 | AGCAGCCTGA TCAGCTATGA GGAGGACCAG AGGCAGGGGG CTGAGCCCAG GAAGAACTTT |
| 3361 | GTGAAGCCCA ATGAAACCAA GACCTACTTC TGGAAGGTGC AGCACCACAT GGCCCCCACC |
| 3421 | AAGGATGAGT TTGACTGCAA GGCCTGGGCC TACTTCTCTG ATGTGGACCT GGAGAAGGAT |
| 3481 | GTGCACTCTG GCCTGATTGG CCCCCTGCTG GTGTGCCACA CCAACACCCT GAACCCTGCC |
| 3541 | CATGGCAGGC AGGTGACTGT GCAGGAGTTT GCCCTGTTCT TCACCATCTT TGATGAAACC |
| 3601 | AAGAGCTGGT ACTTCACTGA GAACATGGAG AGGAACTGCA GGGCCCCCTG CAACATCCAG |
| 3661 | ATGGAGGACC CCACCTTCAA GGAGAACTAC AGGTTCCATG CCATCAATGG CTACATCATG |
| 3721 | GACACCCTGC CTGGCCTGGT GATGGCCCAG GACCAGAGGA TCAGGTGGTA CCTGCTGAGC |
| 3781 | ATGGGCAGCA ATGAGAACAT CCACAGCATC CACTTCTCTG GCCATGTGTT CACTGTGAGG |
| 3841 | AAGAAGGAGG AGTACAAGAT GGCCCTGTAC AACCTGTACC CTGGGGTGTT TGAGACTGTG |
| 3901 | GAGATGCTGC CCAGCAAGGC TGGCATCTGG AGGGTGGAGT GCCTGATTGG GGAGCACCTG |
| 3961 | CATGCTGGCA TGAGCACCCT GTTCCTGGTG TACAGCAACA AGTGCCAGAC CCCCCTGGGC |
| 4021 | ATGGCCTCTG GCCACATCAG GGACTTCCAG ATCACTGCCT CTGGCCAGTA TGGCCAGTGG |
| 4081 | GCCCCCAAGC TGGCCAGGCT GCACTACTCT GGCAGCATCA ATGCCTGGAG CACCAAGGAG |
| 4141 | CCCTTCAGCT GGATCAAGGT GGACCTGCTG GCCCCCATGA TCATCCATGG CATCAAGACC |
| 4201 | CAGGGGGCCA GGCAGAAGTT CAGCAGCCTG TACATCAGCC AGTTCATCAT CATGTACAGC |
| 4261 | CTGGATGGCA AGAAGTGGCA GACCTACAGG GGCAACAGCA CTGGCACCCT GATGGTGTTC |
| 4321 | TTTGGCAATG TGGACAGCTC TGGCATCAAG CACAACATCT TCAACCCCCC CATCATTGCC |
| 4381 | AGATACATCA GGCTGCACCC CACCCACTAC AGCATCAGGA GCACCCTGAG GATGGAGCTG |
| 4441 | ATGGGCTGTG ACCTGAACAG CTGCAGCATG CCCCTGGGCA TGGAGAGCAA GGCCATCTCT |
| 4501 | GATGCCCAGA TCACTGCCAG CAGCTACTTC ACCAACATGT TTGCCACCTG GAGCCCCAGC |
| 4561 | AAGGCCAGGC TGCATCTGCA GGGCAGGAGC AATGCCTGGA GGCCCCAGGT CAACAACCCC |
| 4621 | AAGGAGTGGC TGCAGGTGGA CTTCCAGAAG ACCATGAAGG TGACTGGGGT GACCACCCAG |
| 4681 | GGGGTGAAGA GCCTGCTGAC CAGCATGTAT GTGAAGGAGT TCCTGATCAG CAGCAGCCAG |
| 4741 | GATGGCCACC AGTGGACCCT GTTCTTCCAG AATGGCAAGG TGAAGGTGTT CCAGGGCAAC |
| 4801 | CAGGACAGCT TCACCCCTGT GGTGAACAGC CTGGACCCCC CCCTGCTGAC CAGATACCTG |
| 4861 | AGGATTCACC CCCAGAGCTG GGTGCACCAG ATTGCCCTGA GGATGGAGGT GCTGGGCTGT |
| 4921 | GAGGCCCAGG ACCTGTACTG AGGATCCAAT AAAATATCTT TATTTTCATT ACATCTGTGT |
| 4981 | 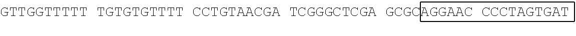 |
| 5041 | 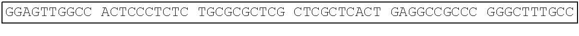 |
| 5101 | 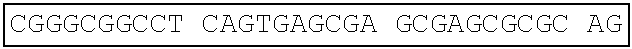 |

In the above sequence, the left (5′) ITR (AAV2 ITR) spans nucleotides 1-130, and the right (3′) ITR (AAV2 ITR) spans nucleotides 5025-5132. Both ITRs are boxed.

### IV. Use of Recombinant AAV Formulations

The pharmaceutical compositions of the invention may be supplied in an article of manufacture (e.g., a kit) comprising vials (e.g., pre-treated glass vials or COP vials) and instructions for use. In some embodiments, each vial contains about 1E+11 to 1E+15 vg per mL of AAV in 0.5-50 mL (e.g., 1-10 mL). In particular embodiments, each vial contains 5E+12 to 1E+14/mL (e.g., 1E+13 vg/mL). In certain embodiments, each vial contains 6E+13 vg in 6 mL.

The compositions may be administered to the patients once or more than once. For example, the compositions may be administered to the patients with an interval of no less than 1 month, 3 months, 6 months, 9 months, or one year. In some embodiments, the composition may be administered to the patients with an interval of no less than two, five, seven, ten, or fifteen years. The pharmaceutical composition may be provided to a patient in need thereof through a route appropriate for the disease to be treated. For example, the compositions may be administered through intravenous injection, intraarterially injection, intracranial injection, intraperitoneal injection, portal vein injection, or intramuscular injection. For example, the SB-525 pharmaceutical compositions may be provided intravenously to hemophilia A patients at 1E+11 to 1E+15 vg/kg, such as 1E+11 to 1E+14 (e.g., 1E+12 to 1E+14) vg/kg. In some embodiments, the SB-525 pharmaceutical compositions may be provided intravenously to hemophilia A patients at 5E+11, 6E+11, 7E+11, 8E+11, 9E+11, 1E+12, 2E+12, 3E+12, 4E+12, 5E+12, 6E+12, 7E+12, 8E+12, 9E+12, 1E+13, 2E+13, 3E+13, 4E+13, 5E+13, 6E+13, 7E+13, 8E+13, 9E+13, or 1E+14 vg/kg. In some embodiments, the SB-525 compositions are provided intravenously to hemophilia A patients at a dose of about 6E+13 vg/kg.

In some embodiments, the patients have with severe or moderate hemophilia A. In further some embodiments, the patients have no inhibitors (alloantibodies to Factor VIII). In certain embodiments, the patients do not have neutralizing antibodies to AAV6. The patients may be adult or adolescent patients (≥12 years of age) or pediatric patients (<12 years of age).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

The following Examples describe a study in which the inventors discovered a current SB-525 formulation formed precipitates upon freeze/thaw cycling, a common test for assessing the shelf-life and long-term stability of biological products. The inventors discovered that precipitates also formed in the buffer formulation alone (i.e., buffer formulation containing no active viral vector ingredient). The inventors thus set out to find a new formulation that would circumvent this problem.

In the study, the SB-525 viral preparations were prepared and formulated at approximately 1.0E+13 vg/mL in phosphate-buffered saline (PBS) with the addition of CaCl₂, MgCl₂, 35 mM NaCl, 1% sucrose, and 0.05% Kolliphor® P 188 (poloxamer 188), filled at 5 mL into 6 mL Aseptic Technologies crystal closed vials, and stored at ≤−65° C. For the stability tests, SB-525 compositions were placed on stability at −0° C., 5° C., and 25° C. for up to 24 months (Table 5). Five cycles of uncontrolled freeze/thaw (F/T) (Table 1) and a 24-hour agitation (AG) (Table 2) were also performed. Drug product (DP) quality attributes were analyzed according to Table 5 to Table 8.

### Example 1: Freeze/Thaw Cycling and Agitation Tests

### Materials and Methods

The SB-525 was provided at 1.0E+13 vg/mL nominal in a formulation buffer containing the following ingredients: 0.90 mM CaCl₂, 0.49 mM MgCl₂, 2.68 mM KCl, 1.47 mM KH₂PO₄, 172 mM NaCl, 8.10 mM Na₂HPO₄, 1% (w/v) sucrose, 0.05% (w/v) poloxamer 188, pH 7.36. 23 vials were stored at −70° C., 11 at 5° C., and 8 at 25° C.

For freeze/thaw (F/T) cycling tests, samples were uncontrolled F/T cycled for three and five cycles as shown in Table 1.

TABLE 1
DP Uncontrolled F/T Cycling

| F/T | Day 1 | Day 2 | | Day 3 | | Day 4 | | Day 5 | |
|---|---|---|---|---|---|---|---|---|---|
| Cycling | 4 pm | 8 am | 4 pm | 8 am | 4 pm | 8 am | 4 pm | 8 am | 4 pm |
| 3 | −80° C. | 25° C. | −80° C. | 25° C. | −80° C. | 25° C. | | | |
| 5 | −80° C. | 25° C. | −80° C. | 25° C. | −80° C. | 25° C. | −80° C. | 25° C. | −80° C. |

For agitation tests, samples were agitated at room temperature as shown in Table 2.

TABLE 2
DP Agitation Setup

| Agitation | T₀ | T_{24Hr} |
|---|---|---|
| Stability | Day 1 (8 am) | Day 2 (8 am) |
| 300 rpm | Use Stability T₀ | Pull 24 Hr material |

The vials stored at −70° C., 5° C., at 25° C., respectively, were analyzed for (i) appearance (liquid), pH, dynamic light scattering (DLS), and high accuracy liquid particle counter (HIAC); (ii) vg identity (qPCR), vg titer (qPCR), capsid titer (ELISA), capsid identity (ELISA), and infectious titer (TCID₅₀); (iii) in vitro FVIII Activity (bioassay); or (iv) UV 260/280, reduced CE-SDS, SEC titer_260/280.

Samples that went through F/T cycling were analyzed for (i) appearance (liquid), pH, osmolarity, DLS, HIAC; (ii) vg titer (qPCR), capsid titer and identity (ELISA), reduced SDS-PAGE, and infectious titer (TCID₅₀); or (iii) UV 260/280, SEC titer_260/280, RP-HPLC, in vitro FVIII Activity (bioassay), and reduced CE-SDS.

Samples that went through 24-hour agitation (AG) were analyzed for (i) appearance (liquid), pH, osmolarity, DLS, and HIAC; (ii) vg titer (qPCR), capsid titer and identity (ELISA), reduced SDS-PAGE, infectious titer (TCID₅₀); or (iii) UV 260/280, SEC titer_260/280, RP-HPLC, in vitro FVIII activity (bioassay), and reduced CE-SDS.

### Results

Results from the SB-525 DP stability, 5 cycles of uncontrolled F/T, and 24-hour agitation arms are described below.

As shown in Table 3 below, there was no significant change in color, clarity, pH, hydrodynamic radius as measured by DLS, vg titer, capsid titer, capsid purity as measured by SDS-PAGE, infectious titer, UV 260/280 (a surrogate measure for empty:full capsid ratio), and biological activity (% relative potency) when the samples were stored at the tested conditions for up to one month. There was a change in visible particles at 5° C. and 25° C. All the samples were frozen and appeared white and opaque at T zero (T₀).

TABLE 3
Stability Results

| Test | | Storage | T₀* | T_{1M}² |
|---|---|---|---|---|
| Appear- | Color | −70° C. | Colorless, clear, | B6 |
| ance, | | 5° C. | no precipitates, | B6 |
| Liquid | | 25° C. | no contaminants | B6 |
| | Clarity | −70° C. | | <= Ref. I |
| | | 5° C. | | <= Ref. I |
| | | 25° C. | | <= Ref. I |
| | Visible | −70° C. | | EFVP |
| | Particles | 5° C. | | 1-5 particles |
| | | 25° C. | | TMTC |
| pH | | −70° C. | 7.3 | 7.3 |
| | | 5° C. | | 7.3 |
| | | 25° C. | | 7.1 |
| DLS | Z-average- | −70° C. | — | 1.70e⁻⁷ |
| | Diffusion | 5° C. | | 1.70e⁻⁷ |
| | Coefficient | 25° C. | | 1.70e⁻⁷ |
| | (cm²/s) | | | |
| | Z-average- | −70° C. | 22.3 (diameter) | 14.7 |
| | Radius | 5° C. | | 14.8 |
| | (nm) | 25° C. | | 14.4 |
| | Peak | −70° C. | | 1.74e⁻⁷ |
| | 1-Diffusion | 5° C. | | 1.69e⁻⁷ |
| | Coefficient | 25° C. | | 1.70e⁻⁷ |
| | (cm²/s) | | | |
| | Peak 1-radius | −70° C. | | 14.2 |
| | (nm) | 5° C. | | 14.6 |
| | | 25° C. | | 14.5 |
| | Peak | −70° C. | | 9.42e⁻¹⁰ |
| | 2-Diffusion | 5° C. | | 1.26e⁻⁹ |
| | Coefficient | 25° C. | | — |
| | (cm²/s) | | | |
| | Peak 2-radius | −70° C. | | 2658.7 |
| | (nm) | 5° C. | | 2261.8 |
| | | 25° C. | | — |
| Vg titer by qPCR | | −70° C. | 8.74E+12 | 9.77E+12 |
| (vg/mL)¹ | | 5° C. | | 1.01E+13 |
| | | 25° C. | | 9.98E+12 |
| Capsid ELISA | | −70° C. | 2.76E+13 | 2.52E+13 |
| (capsids/mL) | | 5° C. | | 2.53E+13 |
| | | 25° C. | | 2.66E+13 |
| SDS-PAGE | | −70° C. | VP1, VP2, and | VP1, VP2, and |
| | | 5° C. | VP3 comparable | VP3 comparable to |
| | | 25° C. | to reference. No | reference. No other |
| | | | other bands ≥ | bands ≥ |
| | | | sensitivity control | sensitivity control |
| | | | observed (% | observed (% Purity |
| | | | Purity VP1, VP2, | VP1, VP2, VP3 |
| | | | VP3 combined) = | combined) = 100% |
| | | | 100% | |
| Infectious Titer TCID50 | | −70° C. | 1.12E+9 | 3.56E+9 |
| (TCID₅₀/mL) | | 5° C. | | 2.00E+9 |
| | | 25° C. | | 1.12E+9 |
| SEC UV 260/280 Ratio | | −70° C. | 1.37 | 1.36 |
| | | 5° C. | | 1.36 |
| | | 25° C. | | 1.36 |

| | | | |
|---|---|---|---|
| In vitro FVIII Activity/ | −70° C. | 85³ | 80 |
| Functional Bioassay | 5° C. | | 80 |
| (% Relative Potency) | 25° C. | | 69 |

*T₀: all analytics, except functional bioassay, performed per release testing
¹T₀ re-test of Vector Genome Identity & Titer by qPCR was performed
²Study terminated after T1M due to particle detection in DP; investigation was initiated
³T₀ re-test for Bioassay (% Relative Potency) against a different reference material
EFVP: Essentially Free of Visible Particles
TMTC: Too many to Count
N.T.: Not tested

As shown in Table 4, there was no significant change in capsid purity after one month at all storage conditions.

TABLE 4
Capsid Purity Stability Results

| Time Point | Storage | | Capsid (%) | VP1 (%) | VP2 (%) | VP3 (%) | Other (%) |
|---|---|---|---|---|---|---|---|
| T₀ | | | 94.3 | 2.2 | 3.4 | 94.4 | 5.7 |
| T_{1 M} | −70° | C. | 95.1 | 1.1 | 3.1 | 95.8 | 4.9 |
| | 5° | C. | 95.0 | 2.1 | 3.2 | 94.7 | 5.0 |
| | 25° | C. | 94.8 | 1.9 | 3.0 | 95.1 | 5.2 |

As shown in Table 5, there was no significant aggregation or formation of high molecular weight species (HMWS) after one month at all storage conditions (NMT: not more than; LOQ: limit of quantification).

TABLE 5
SEC Titer Stability Results After One Month
Under Intended, Accelerated, and Stressed Storage Conditions

| Time | | Viral Particle (vp) | Monomer | HMWS | |
|---|---|---|---|---|---|
| Point | Storage | Titer (vp/mL) | (%) | (%) | LOQ |
| T₀ | | 5.12E+13 | 100 | NMT 1.0 | 1.01E+12 |
| T_{1M} | −70° C. | 2.65E+13 | 100 | NMT 1.0 | 1.01E+12 |
| | 5° C. | 2.57E+13 | 100 | NMT 1.0 | 1.01E+12 |
| | 25° C. | 3.31E+13 | 100 | NMT 1.0 | 1.01E+12 |

As shown in Table 6, there was no significant increase in subvisible particles (per HIAC) after one month at all storage conditions. All time points showed particles below the USP <787> standard in regard to the ≥10 μm and the ≥25 μm ranges, where acceptance criteria are no more than 6,000 particles ≥10 μm per container and no more than 600 particles ≥25 μm per container.

TABLE 6
HIAC Stability Results

| | | T₀ (particles/container) | | T_{1 M} (particles/mL) | | | | |
|---|---|---|---|---|---|---|---|---|
| Storage | | ≥10 μm | ≥25 μm | ≥2 μm | ≥5 μm | ≥8 μm | ≥10 μm | ≥25 μm |
| −70° | C. | 10 | 3 | 143 | 11 | 1 | 0 | 0 |
| 5° | C. | | | 626 | 96 | 24 | 15 | 0 |
| 25° | C. | | | 478 | 94 | 15 | 4 | 1 |

As shown in Table 7, there was no significant change in color, clarity, pH, hydrodynamic radius as measured by DLS, vg titer, capsid titer, infectious titer, UV 260/280 (surrogate for empty:full capsid ratio), capsid purity as measured by RP-HPLC, and biological activity (% relative potency) upon F/T/AG stress. There was a change in visible particles for F/T.

TABLE 7
Cycle Uncontrolled F/T & 24-Hour AG Results

| Test | | Treatment | Results |
|---|---|---|---|
| Appearance | Color | F/T | B9¹ |
| | | AG | B9 |
| | Clarity | F/T | <= REF_1² |
| | | AG | <= REF_1 |
| | Visible Particles | F/T | TMTC |
| | | AG | Essentially free of visible |
| | | | particles (EFVP) |
| pH | | F/T | 7.2 |
| | | AG | 7.3 |
| Osmolality (mOsm/kg) | | F/T | 387 |
| | | AG | 388 |
| DLS | Z-average-Diffusion | F/T | 1.70e⁻⁷ |
| | Coefficient (cm²/s) | AG | 1.67e⁻⁷ |
| | Z-average-Radius | F/T | 14.5 |
| | (nm) | AG | 14.7 |
| | Peak 1-Diffusion | F/T | 1.69e⁻⁷ |
| | Coefficient (cm²/s) | AG | 1.56e⁻⁷ |
| | Peak 1-radius (nm) | F/T | 14.5 |
| | | AG | 15.7 |
| Vg titer by qPCR (vg/mL) | | F/T | 8.44E+12 |
| | | AG | 8.63E+12 |
| Capsid ELISA (capsids/mL) | | F/T | 2.08E+13 |
| | | AG | 2.00E+13 |
| SDS-PAGE | | F/T | VP1, VP2, and |
| | | AG | VP3 comparable to |
| | | | reference. No other |
| | | | bands ≥ sensitivity |
| | | | control observed |
| | | | (% Purity VP1, VP2, |
| | | | VP3 combined) = 100% |
| Infectious Titer TCID50 | | F/T | 9.29E+8 |
| (TCID₅₀/mL) | | AG | 2.42E+9 |
| UV 260/280 Ratio | | F/T | 1.32 |
| | | AG | 1.32 |
| SEC UV 260/260 Ratio | | F/T | 1.35 |
| | | AG | 1.35 |
| RP-HPLC | Capsid (%) | F/T | 98.9 |
| | Other (%) | AG | 99.0 |
| | | F/T | 1.1 |
| | | AG | 1.0 |

| | | |
|---|---|---|
| in vitro FVIII Activity/Functional | F/T | 85 |
| Bioassay | AG | 80 |
| (% Relative Potency) | | |

¹Color is not more intense or more opalescent than reference standard listed.
²Clarity is not more opalescent than reference standard listed.

As shown in Table 8, there was no significant change in reduced CE-SDS upon F/T/AG stress.

TABLE 8
5 Cycle Uncontrolled F/T & 24-Hour AG CE-SDS-Reduced Results

| Treatment | Capsid (%) | VP1 (%) | VP2 (%) | VP3 (%) | Other (%) |
|---|---|---|---|---|---|
| F/T | 95.1 | 2.0 | 3.0 | 95.0 | 4.9 |
| AG | 95.2 | 2.0 | 3.2 | 94.8 | 4.8 |

As shown in Table 9, there was no significant aggregation/HMWS formation upon F/T/AG stress.

TABLE 9
5 Cycle Uncontrolled F/T & 24-Hour AG SEC Titer Results

| | Viral Particle Titer | Monomer | HMWS | |
|---|---|---|---|---|
| Treatment | (vp/mL) | (%) | (%) | LOQ |
| F/T | 4.95E+13 | 100 | 0 | 1.01E+12 |
| AG | 5.05E+13 | 100 | 0 | 1.01E+12 |

As shown in Table 10, there was a significant increase in subvisible particles (per HIAC) upon F/T. Particles upon F/T were above USP <787> standard with regard to the ≥10 μm and the ≥25 μm ranges; whereas particles upon AG were below USP <787> standard with regard to the ≥10 μm and the ≥25 μm ranges.

TABLE 10
5 Cycle Uncontrolled F/T & 24-Hour AG HIAC Results

| | ≥2 μm | ≥5 μm | ≥8 μm | ≥10 μm | ≥25 μm |
|---|---|---|---|---|---|
| Treat- | (particles/ | (particles/ | (particles/ | (particles/ | (particles/ |
| ment | mL) | mL) | mL) | mL) | mL) |
| F/T | 24551 | 14051 | 8725 | 6576 | 710 |
| AG | 439 | 41 | 12 | 4 | 0 |

The above results demonstrate that the SB-525 samples showed no significant change in color, clarity, pH, hydrodynamic radius (as measured by dynamic light scattering), vg titer, capsid titer, infectious titer, empty:full capsid ratio (as measured by UV 260/280), biological activity (% relative potency), capsid purity (as measured by SDS-PAGE, reduced CE-SDS and RP-HPLC), or viral particle titer when stored up to one month at −70° C., 5° C., or 25° C. Additionally, there was no significant change in color, clarity, pH, hydrodynamic radius (as measured by dynamic light scattering), vg titer, capsid titer, infectious titer, empty:full capsid ratio (as measured by UV 260/280), biological activity (% relative potency), capsid purity (as measured by SDS-PAGE, reduced CE-SDS, RP-HPLC), or viral particle titer upon F/T or AG stress, either. However, there was a significant increase in subvisible particles (per HIAC) upon F/T and an increase in visible particles after one month of storage at 5° C. and 25° C. The study was terminated at one month because of these results.

### Example 2: Characterization of Particulate Matter in DP Vials After F/T

The samples from the above stability tests of SB-525 formulations, in which particulate matter was observed, were analyzed further for isolation and identification.

Drug product samples that had undergone 5 freeze-thaw cycles (“DP after 5 F/T”) and samples that had been stored at 25° C. for one month (DP after 1M at 25° C.) were analyzed. Particulate matter was isolated onto 0.8 μm gold filters. It was then imaged on the filter under partial ring lighting using a Keyence VHX6000 digital microscope at 150× magnification. The filters were then transferred to the Fourier transform infrared (FTIR) microscope where spectra were obtained for the material. FTIR spectra were compared against the KnowItAll database for known spectra. A portion of the DP after 1M at 25° C. sample was also scraped off the filter onto a glass slide and imaged under plane polarized light and crossed polars using a Nikon Eclipse ME600 polarized light microscope. A portion of the gold filter was cut and analyzed under the JEOL6000 SEM equipped with an EDS module. The sample was also analyzed by Raman microscopy analysis.

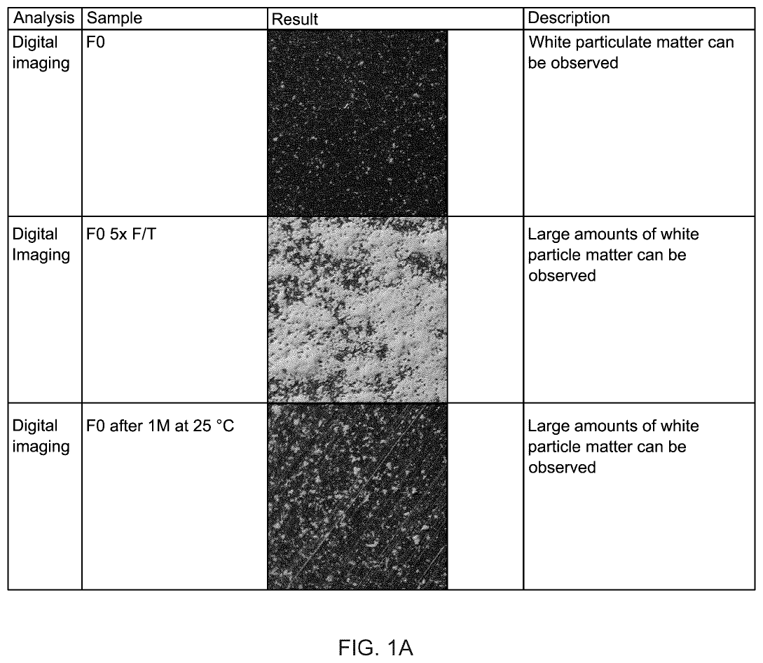

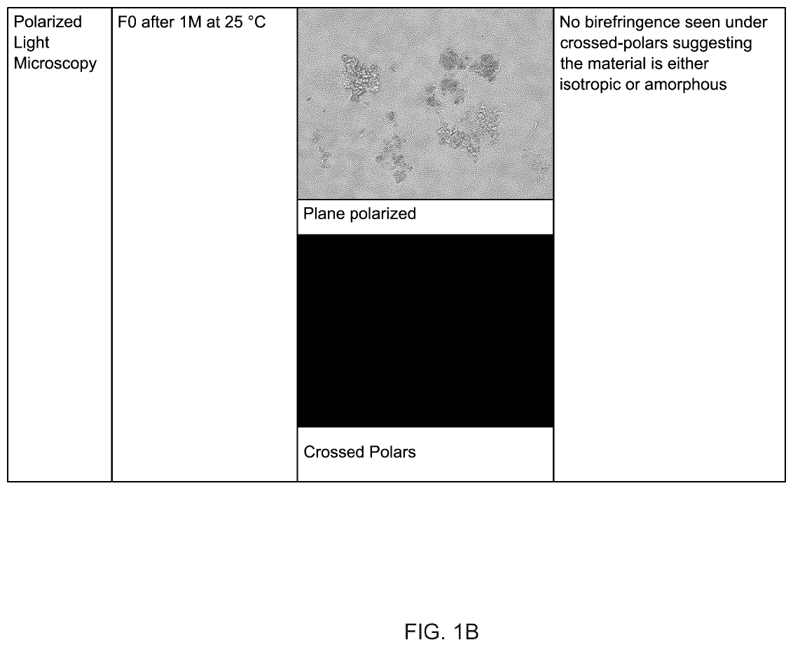

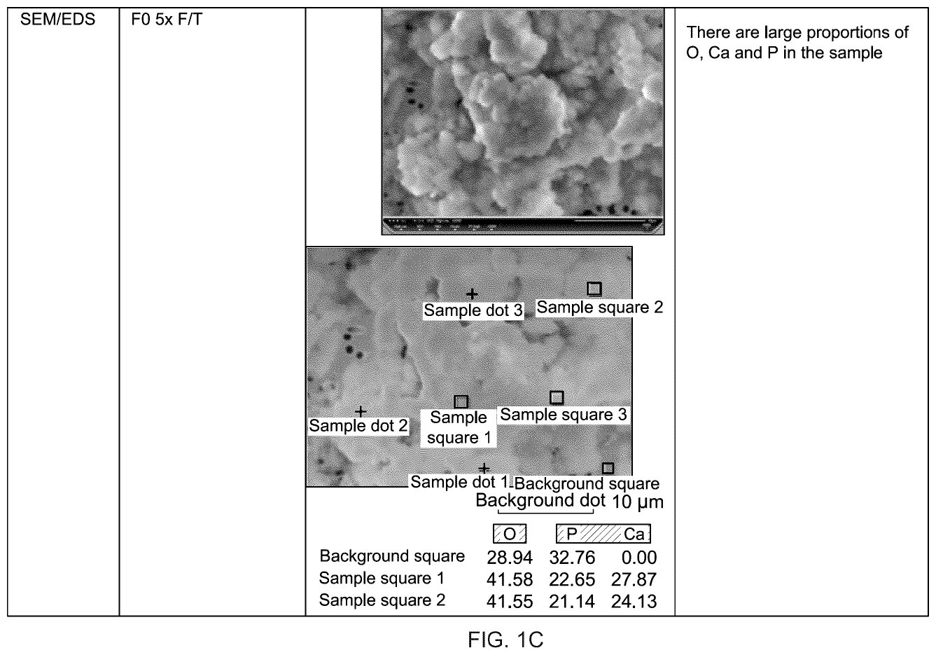

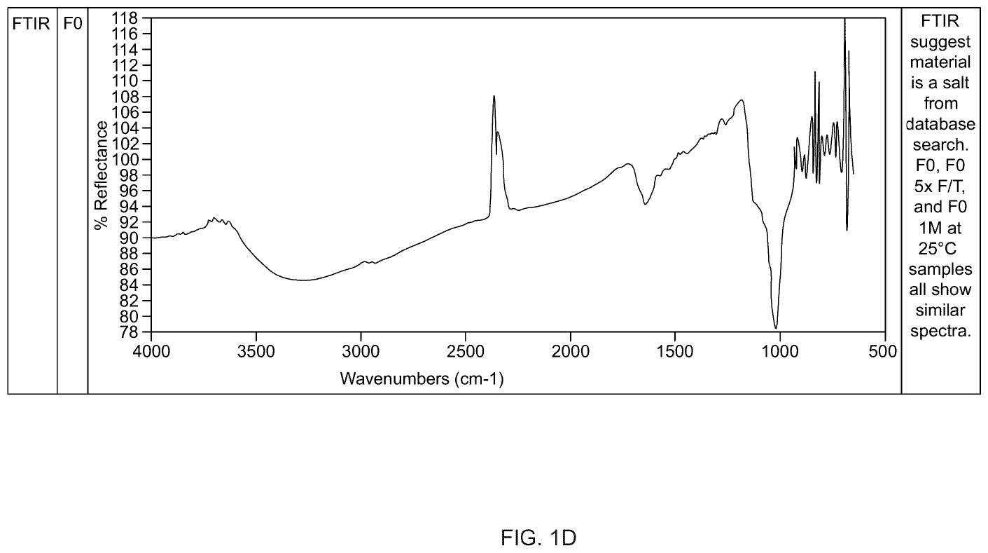

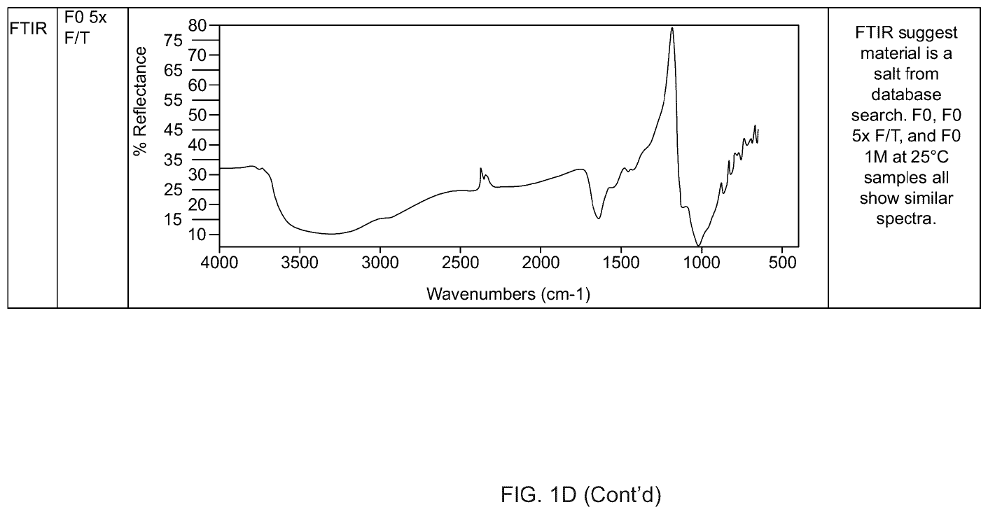

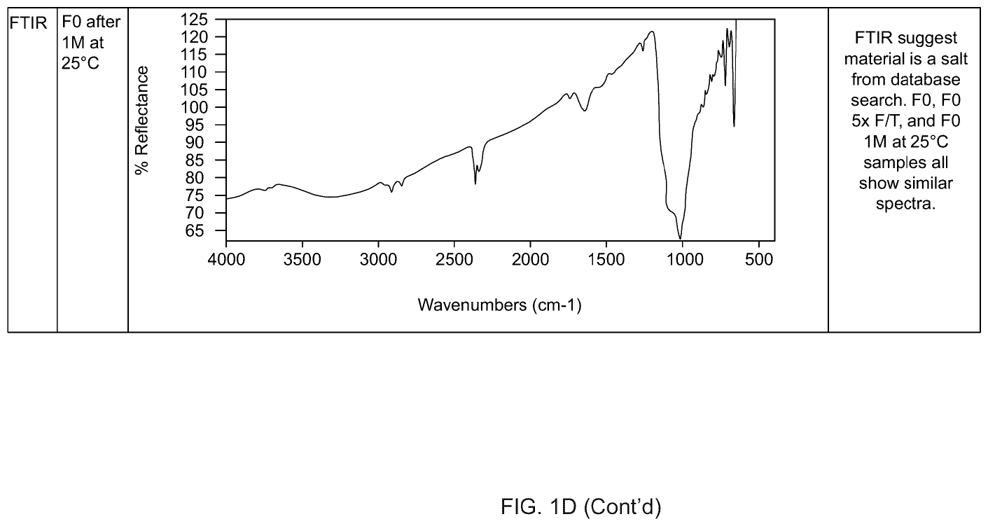

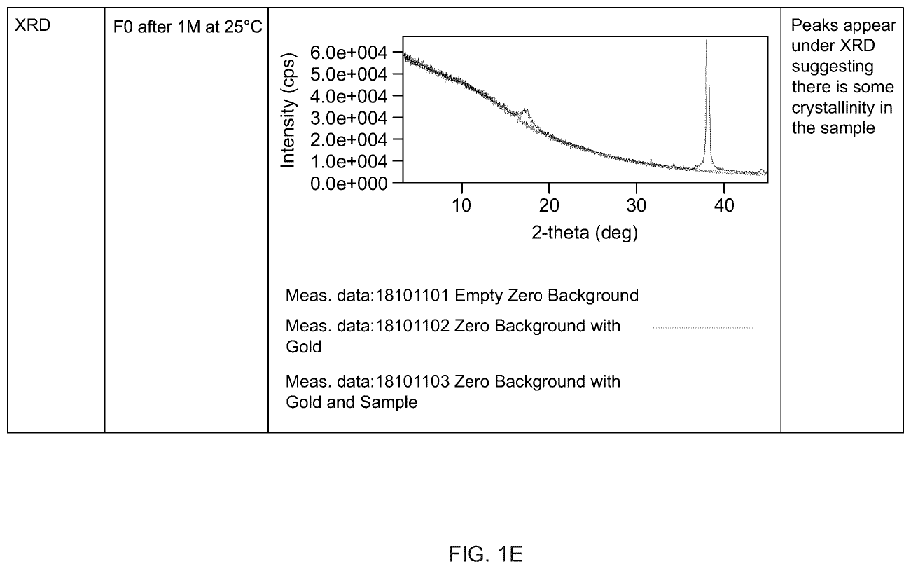

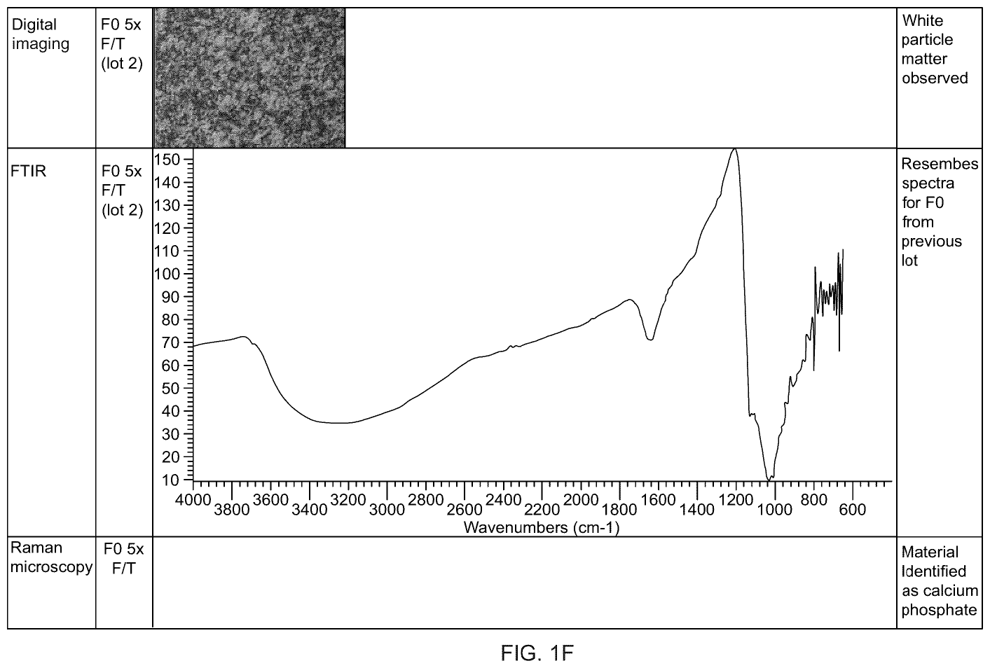

For all samples, digital imaging showed a white solid that appeared to be semi-crystalline. Under crossed polars, the “DP after 5 F/T” sample did not exhibit any birefringence, suggesting it was either an amorphous substance or isotropic. SEM/EDS analysis showed large portions of oxygen, phosphorous, and calcium. FTIR analysis identified the material as a salt, but further identification was required. Raman microscopy was able to detect peaks characteristic of calcium phosphate, suggesting the white particulate matter in all samples was calcium phosphate. FIGS. 1A-F summarize the particle characterization studies.

### Example 3: Reformulation of SB-525

This example describes experiments testing new SB-525 formulations that can circumvent the precipitation problem observed above. These experiments assessed the short-term stability of SB-525 DP when reformulated to (1) remove calcium, (2) remove calcium and magnesium, or (3) increase sucrose to 8.5%. These changes were expected to prevent generation of particles by (1) removing the source of particles, (2) removing both divalent cations due to solubility concerns of MgCl₂, or (3) increasing stability of the formulation against freeze-thaw stress, respectively. Two pilot batches were buffer exchanged and filled at 2.5 mL into 6 mL AT vials to mimic worst-case conditions for surface area to volume (SA/V) fill. After fill/finish, vials were subjected to 5 uncontrolled freeze-thaw cycles (≤−65° C. to ambient), and then placed on stability at ≤−65° C. (intended storage), 2-8° C. (stressed; liquid storage), 25° C. (accelerated; liquid storage), and 40° C. (aggressive/forced degradation condition; liquid storage). These conditions were selected with the expectation of highlighting differences between formulations.

SB-525 virus was purified from the clarified bulk harvest and formulated at approximately 1.0E+13 vg/mL in phosphate buffered saline (PBS) containing CaCl₂, MgCl₂, 35 mM NaCl, 1% Sucrose, and 0.05% Kolliphor® P 188 (poloxamer 188), filled into vials, and stored at ≤−65° C.

### Materials and Methods

The SB-525 formulated bulk drug substance contained: SB-525 (1.0E+13 vg/ mL nominal) in 0.90 mM CaCl₂, 0.49 mM MgCl₂, 2.68 mM KCl, 1.47 mM KH₂PO₄, 172 mM NaCl, 8.10 mM Na₂HPO₄, 1% (w/v) sucrose, 0.05% (w/v) poloxamer 188, pH 7.0-7.6, sterile filtered and stored in 125 mL HDPE bottles at −65° C. Na₂HPO₄ (sodium phosphate, dibasic, anhydrous), KH₂PO₄ (potassium phosphate monobasic), CaCl₂ (calcium chloride dihydrate), and MgCl₂ (magnesium chloride hexahydrate) were sourced from JT Baker or Fischer. Poloxamer 188 was sourced from BASF. Storage vials were 6 mL Aseptic Technologies (AT) Closed Crystal Vials (Aseptic Technologies; cat #VIA-060000), which are vials with primary container closure, cyclo-olefin copolymer (COC) with in-place thermoplastic elastomer stopper and yellow cap. Prior to DP fill, the formulated bulk drug substance (FBDS) was filled into 125 mL high density polyethylene (HDPE) bottles and frozen to ≤−65° C. Bottles were then thawed at ambient temperature, buffer exchanged, filtered, and DP filled into 6 mL AT vials using the Aseptic Technologies M1 unit. One pilot batch material was buffer exchanged via TFF using 50 kDa filter units at a pressure of approximately 40 psi for a total of 10 exchange volumes. The second pilot batch was buffer exchanged using Amicon stir cells over a 50 kDa NMW PES filter at approximately 40 psi for a total of 10 exchange volumes. The two exchange methods have been previously used for AAV without significant loss or adsorption of material.

The AT vials were filled 2.5 mL at a target of approximately 1.0E+13 vg/mL, freeze thaw cycled 5 times at an uncontrolled rate from ≤−65 ° C. to ambient conditions, and placed on stability in non-GMP storage units. Vials were subjected to the stability pull schedule outlined in Table 11, and tested by the methods listed in Table 12. Formulations tested are outlined in Table 13.

TABLE 11
Capsid Purity Stability Schedule

| Storage Temp (° C.) | | T₀ | T_{3 D} | T_{1 W} | T_{2 W} | T_{3 W} | T_{4 W} | T_{5 W} |
|---|---|---|---|---|---|---|---|---|
| −70 | (±10) | XABCD | — | — | — | — | XABC | XA |
| 5 | (±3) | | X | X | XA | X | | XA |
| 25 | (±3) | | X | X | XAB | X | | — |
| 40 | (±3) | | XABCF | XA | X | XF | — | — |

TABLE 12
Analytical Testing

| Test Code | Test Method |
|---|---|
| X | Appearance (Liquid) |
| A | pH |
| A | SEC-Aggregates, UV 260/280, Viral Particle Titer |
| B | Functional FVIII Bioassay |
| C | Light Obscuration with HIAC |
| D | Osmolality (freezing point depression) |
| D | P188 |
| D | Sucrose |
| D | Reduced capillary gel electrophoresis |
| | (rCGE) (ratio and purity) |
| F | Mass Spec (deamidation) |
| B | Vector Genome Titer (qPCR) |

TABLE 13
Formulations

| Exchange | | | |
|---|---|---|---|
| Process | Material | Formulation | Difference from Control |
| Millipore | M05 | Control | N/A |
| Amicon Stir | M06 | Ca Negative | Removed CaCl₂ |
| Cell | M07 | Ca/Mg Negative | Removed CaCl₂ and MgCl₂ |
| | M08 | 8.5% Sucrose | Increase sucrose to 8.5% (w/v) |

*Control formulation (F0): 0.90 mM CaCl₂, 0.49 mM MgCl2, 2.68 mM KCl, 1.47 mM KH₂PO₄, 172 mM NaCl, 8.10 mM Na₂HPO₄, 1% (w/v) sucrose, 0.05% (w/v) poloxamer 188 (P188), pH 7.0-7.6.

### Results

### Formulation Characterization

To confirm that the correct formulations were achieved, SB-525 DP formulations were tested to determine osmolality, P188 concentration, sucrose concentration, and capsid ratio and purity by reduced CGE. The results to these tests for M05-M08 are shown in Table 14 below. Capsid purity and ratio showed no significant differences as measured by rCGE. Osmolality is within expected ranges, dependent on the level of sucrose. M05-M08 samples demonstrated the correct concentrations for P188. In addition, concentrations of calcium and magnesium were confirmed by CEDEX to be within expected ranges for all formulations.

TABLE 14
Formulation Characterization

| | Osmolality | Sucrose | P188 | Purity | AAV Ratio | | |
|---|---|---|---|---|---|---|---|
| Material | (mOsm/kg) | (%) | (μg/mL) | (% Capsid) | % VP1 | % VP2 | % VP3 |
| M05 - Control | 399 | 1.10 | 589 | 95.2 | 2.0 | 3.3 | 94.7 |
| M06 -Ca Negative | 391 | 1.00 | 565 | 95.3 | 2.1 | 3.0 | 94.9 |
| M07 - Ca/Mg Negative | 417 | 1.00 | 575 | 95.8 | 1.7 | 2.9 | 95.4 |
| M08 - 8.5% Sucrose | 655 | 8.50 | 568 | 95.2 | 1.8 | 2.6 | 95.6 |

Vector Genome Titer (vg/mL)

Vector genome titer results for M05-M08 are presented below in Table 15. At ≤−65° C. and 2-8° C. conditions, there was no significant trend observed after storage for 4 weeks. After 40° C. for 3 days, the Ca/Mg negative formulation showed a downward trend in vector genome titer. This result is considered within assay variability, but is consistent with vp titer and UV 260/280 results for this formulation.

TABLE 15
Genome Titer (vg/mL)

| | Timepoint | | | | | |
|---|---|---|---|---|---|---|
| Formulation | T₀ | T_{3 D} | T_{1 W} | T_{2 W} | T_{3 W} | T_{4 W} |

−70° C.

| M05 - Control | 7.44E+12 | Not tested | 7.77E+12 |
|---|---|---|---|
| M06 - Ca Negative | 7.98E+12 | | 7.47E+12 |
| M07 - Ca/Mg Negative | 6.33E+12 | | 6.24E+12 |
| M08 - 8.5% Sucrose | 6.23E+12 | | 6.56E+12 |

5° C.

| M05 - Control | 7.44E+12 | Not tested | 7.39E+12 |
|---|---|---|---|
| M06 - Ca Negative | 7.98E+12 | | 6.95E+12 |
| M07 - Ca/Mg Negative | 6.33E+12 | | 6.56E+12 |
| M08 - 8.5% Sucrose | 6.23E+12 | | 6.24E+12 |

25° C.

| M05 - Control | 7.44E+12 | Not tested | 7.23E+12 | Not tested | 7.84E+12 |
|---|---|---|---|---|---|
| M06 - Ca Negative | 7.98E+12 | | 7.41E+12 | | 7.09E+12 |
| M07 - Ca/Mg Negative | 6.33E+12 | | 6.04E+12 | | 5.85E+12 |
| M08 - 8.5% Sucrose | 6.23E+12 | | 6.11E+12 | | 7.01E+12 |

40° C.

| M05 - Control | 7.44E+12 | 7.16E+12 | Not tested |
|---|---|---|---|
| M06 - Ca Negative | 7.98E+12 | 6.94E+12 | |
| M07 - Ca/Mg Negative | 6.33E+12 | 4.63E+12 | |
| M08 - 8.5% Sucrose | 6.23E+12 | 6.68E+12 | |

### Visible Appearance

All formulations displayed B9 color and less than or equal to opalescence reference 1, color and clarity standards, respectively, used during visual inspection against a black and white background, for all timepoints and conditions (see, e.g., Ph. Eur. 7.0, 20201, 20202 (01/2008); Millipore Sigma Colour Reference Solutions B). Visible appearance results show that only the “control” formulation (M05) displayed white flake-like particles that were too many to count (TMTC) when held at 25° C. for longer than 1 week, or 40° C. for 3 days or longer. M06 and M07 formulations did occasionally show TMTC visible particles; however, the particle descriptions for these samples were all fiber-like particles. While these particles were not identified for this study, fiber-like particles are more characteristic of exogenous particles (e.g., filter particles) versus the flake-like particles that are characteristic of calcium phosphate. Also, appearance of fiber-like/exogenous particles is negligible for lab-based development studies where DP is not manufactured under tightly controlled environmental conditions.

### Cumulative Sub-Visible Particles

Sub-visible cumulative particle analysis by the HIAC showed the only formulation with significant sub-visible particle count was the “control” formulation, which showed an increasing trend with more aggressive stability conditions (higher temperatures for longer time). Other formulations (M06-M08) showed only non-significant particle counts, independent of time or condition.

### Functional Bioassay (Potency)

Functional FVIII bioassay results are presented below in Table 16. Results showed no significant changes in potency when samples are kept at either ≤−65° C. or 2-8° C. conditions. At 25° C., there was a downward trend in potency for all formulations (M05-M08) after 4 weeks. At 40° C., there was a significant drop in potency for the Ca/Mg negative formulation after 3 days.

TABLE 16
Functional Bioassay (Relative Potency)

| | | Timepoint | | | | | |
|---|---|---|---|---|---|---|---|
| Formulation | Material | T₀ | T_{3 D} | T_{1 W} | T_{2 W} | T_{3 W} | T_{4 W} |

−70° C.

| M05 - Control | M05 | 75 | Not tested | 81 |
|---|---|---|---|---|
| M06 - Ca Negative | M06 | 90 | | 81 |
| M07 - Ca/Mg Negative | M07 | 67 | | 68 |
| M08 - 8.5% Sucrose | M08 | 74 | | 74 |

5° C.

| M05 - Control | M05 | 75 | Not tested | 73 |
|---|---|---|---|---|
| M06 - Ca Negative | M06 | 90 | | 87 |
| M07 - Ca/Mg Negative | M07 | 67 | | 71 |
| M08 - 8.5% Sucrose | M08 | 74 | | 76 |

25° C.

| M05 - Control | M05 | 75 | Not tested | 53 | Not tested | 37 |
|---|---|---|---|---|---|---|
| M06 - Ca Negative | M06 | 90 | | 66 | | 63 |
| M07 - Ca/Mg Negative | M07 | 67 | | 39 | | 41 |
| M08 - 8.5% Sucrose | M08 | 74 | | 55 | | 45 |

40° C.

| M05 - Control | M05 | 75 | 51 | Not tested |
|---|---|---|---|---|
| M06 - Ca Negative | M06 | 90 | 60 | |
| M07 - Ca/Mg Negative | M07 | 67 | 22 | |
| M08 - 8.5% Sucrose | M08 | 74 | 56 | |

### SEC

SEC results are presented below. Percent aggregate results are shown in Table 17 below. No significant trend in aggregation was seen at ≤−65° C., 2-8° C., or 25° C. stability conditions. At 40° C., there was an increasing trend in aggregates for the Ca/Mg negative formulation, up to 7% HMWS after one week. The “calcium negative” formulation showed approximately 3% HMWS at the end of one week at 40° C. Other formulations did not show significant growth in aggregation when held at ≤−65° C. or 2-8° C. for up to 5 weeks, 25° C. for up to four weeks, and 40° C. for up to 1 week.

TABLE 17
Concentration of Aggregates by SEC (% HMWS)

| | | Timepoint | | | | | | |
|---|---|---|---|---|---|---|---|---|
| Formulation | Material | T₀ | T_{3 D} | T_{1 W} | T_{2 W} | T_{3 W} | T_{4 W} | T_{5 W} |

−70° C.

| M05 - Control | M05 | NMT | NT | NMT | NMT |
|---|---|---|---|---|---|
| | | 1.0 | | 1.0 | 1.0 |
| M06 - Ca | M06 | NMT | | NMT | NMT |
| Negative | | 1.0 | | 1.0 | 1.0 |
| M07 - Ca/Mg | M07 | NMT | | NMT | NMT |
| Negative | | 1.0 | | 1.0 | 1.0 |
| M08 - 8.5% | M08 | NMT | | NMT | NMT |
| Sucrose | | 1.0 | | 1.0 | 1.0 |

5° C.

| M05 - Control | M05 | NMT | NT | NMT | NT | NMT | NMT |
|---|---|---|---|---|---|---|---|
| | | 1.0 | | 1.0 | | 1.0 | 1.0 |
| M06 - Ca | M06 | NMT | | NMT | | NMT | NMT |
| Negative | | 1.0 | | 1.0 | | 1.0 | 1.0 |
| M07 - Ca/Mg | M07 | NMT | | NMT | | NMT | NMT |
| Negative | | 1.0 | | 1.0 | | 1.0 | 1.0 |
| M08 - 8.5% | M08 | NMT | | NMT | | NMT | NMT |
| Sucrose | | 1.0 | | 1.0 | | 1.0 | 1.0 |

25° C.

| M05 - Control | M05 | NMT | NT | NMT | NT | NMT | NT |
|---|---|---|---|---|---|---|---|
| | | 1.0 | | 1.0 | | 1.0 | |
| M06 - Ca | M06 | NMT | | NMT | | NMT | |
| Negative | | 1.0 | | 1.0 | | 1.0 | |
| M07 - Ca/Mg | M07 | NMT | | NMT | | 2.6 | |
| Negative | | 1.0 | | 1.0 | | | |
| M08 - 8.5% | M08 | NMT | | NMT | | NMT | |
| Sucrose | | 1.0 | | 1.0 | | 1.0 | |

40° C.

| M05 - Control | M05 | NMT | NMT | NMT | NT |
|---|---|---|---|---|---|
| | | 1.0 | 1.0 | 1.0 | |
| M06 - Ca | M06 | NMT | 3.1 | 2.6 | |
| Negative | | 1.0 | | | |
| M07 - Ca/Mg | M07 | NMT | 5.3 | 7.0 | |
| Negative | | 1.0 | | | |
| M08 - 8.5% | M08 | NMT | 1.9 | NMT | |
| Sucrose | | 1.0 | | 1.0 | |

*NMT: no more than. NT: Not tested.

Viral particle results are shown below in Table 18. All formulations held at ≤−65° C., 2-8° C., or 25° C. conditions showed no significant change in viral particle titer. However, at 40° C., there was a significant drop in particle titer for the Ca/Mg negative formulation after 3 days and 1 week. Other formulations (M05-M06, M08) did not show a significant drop in viral particle titer when held at ≤−65° C. or 2-8° C. for up to 5 weeks, 25° C. for up to four weeks, and 40° C. for up to 1 week.

TABLE 18
Viral Particle Titer by SEC (vp/mL)

| | | Timepoint | | | | | | |
|---|---|---|---|---|---|---|---|---|
| Formulation | Material | T₀ | T_{3 D} | T_{1 W} | T_{2 W} | T_{3 W} | T_{4 W} | T_{5 W} |

−70° C.

| M05 - | M05 | 2.46E+13 | NT | 2.43E+13 | 2.54E+13 |
|---|---|---|---|---|---|
| Control | | | | | |
| M06 - | M06 | 2.44E+13 | | 2.41E+13 | 2.61E+13 |
| Ca Negative | | | | | |
| M07 - | M07 | 2.02E+13 | | 2.01E+13 | 2.11E+13 |
| Ca/Mg | | | | | |
| Negative | | | | | |
| M08 - | M08 | 2.13E+13 | | 2.16E+13 | 2.27E+13 |
| 8.5% Sucrose | | | | | |

5° C.

| M05 - | M05 | 2.46E+13 | NT | 2.55E+13 | NT | 2.45E+13 | 2.51E+13 |
|---|---|---|---|---|---|---|---|
| Control | | | | | | | |
| M06 - | M06 | 2.44E+13 | | 2.54E+13 | | 2.41E+13 | 2.62E+13 |
| Ca Negative | | | | | | | |
| M07 - | M07 | 2.02E+13 | | 2.11E+13 | | 1.99E+13 | 2.14E+13 |
| Ca/Mg | | | | | | | |
| Negative | | | | | | | |
| M08 - | M08 | 2.13E+13 | | 2.25E+13 | | 2.16E+13 | 2.34E+13 |
| 8.5% Sucrose | | | | | | | |

25° C.

| M05 - | M05 | 2.46E+13 | NT | 2.49E+13 | NT | 2.35E+13 | NT |
|---|---|---|---|---|---|---|---|
| Control | | | | | | | |
| M06 - | M06 | 2.44E+13 | | 2.53E+13 | | 2.39E+13 | |
| Ca Negative | | | | | | | |
| M07 - | M07 | 2.02E+13 | | 2.01E+13 | | 1.90E+13 | |
| Ca/Mg | | | | | | | |
| Negative | | | | | | | |
| M08 - | M08 | 2.13E+13 | | 2.23E+13 | | 2.11E+13 | |
| 8.5% Sucrose | | | | | | | |

40° C.

| M05 - | M05 | 2.46E+13 | 2.23E+13 | 2.14E+13 | NT |
|---|---|---|---|---|---|
| Control | | | | | |
| M06 - | M06 | 2.44E+13 | 2.16E+13 | 2.09E+13 | |
| Ca Negative | | | | | |
| M07 - | M07 | 2.02E+13 | 1.62E+13 | 1.59E+13 | |
| Ca/Mg | | | | | |
| Negative | | | | | |
| M08 - | M08 | 2.13E+13 | 2.09E+13 | 2.06E+13 | |
| 8.5% Sucrose | | | | | |

UV 260/280 ratio results are presented in Table 19 below. For UV 260/280 ratio, there was a recorded downward trend for the Ca/Mg negative formulation when held at 40° C. UV 260/280 is considered a surrogate for empty/full particle ratio. Other formulations (M05, M06, and M08) did not show any significant change in UV 260/280 ratio at any condition or timepoint, as shown.

TABLE 19
UV 260/280 Ratio by SEC

| | | Timepoint | | | | | | |
|---|---|---|---|---|---|---|---|---|
| Formulation | Material | T₀ | T_{3 D} | T_{1 W} | T_{2 W} | T_{3 W} | T_{4 W} | T_{5 W} |

−70° C.

| M05 - Control | M05 | 1.33 | NT | 1.33 | 1.33 |
|---|---|---|---|---|---|
| M06 - Ca Negative | M06 | 1.32 | | 1.32 | 1.33 |
| M07 - Ca/Mg Negative | M07 | 1.32 | | 1.32 | 1.33 |
| M08 - 8.5% Sucrose | M08 | 1.32 | | 1.32 | 1.33 |

5° C.

| M05 - Control | M05 | 1.33 | NT | 1.33 | NT | 1.32 | 1.38 |
|---|---|---|---|---|---|---|---|
| M06 - Ca Negative | M06 | 1.32 | | 1.33 | | 1.33 | 1.33 |
| M07 - Ca/Mg Negative | M07 | 1.32 | | 1.33 | | 1.32 | 1.33 |
| M08 - 8.5% Sucrose | M08 | 1.32 | | 1.33 | | 1.32 | 1.33 |

25° C.

| M05 - Control | M05 | 1.33 | NT | 1.34 | NT | 1.33 | NT |
|---|---|---|---|---|---|---|---|
| M06 - Ca Negative | M06 | 1.32 | | 1.32 | | 1.33 | |
| M07 - Ca/Mg Negative | M07 | 1.32 | | 1.33 | | 1.33 | |
| M08 - 8.5% Sucrose | M08 | 1.32 | | 1.32 | | 1.32 | |

40° C.

| M05 - Control | M05 | 1.33 | 1.33 | 1.33 | NT |
|---|---|---|---|---|---|
| M06 - Ca Negative | M06 | 1.32 | 1.32 | 1.32 | |
| M07 - Ca/Mg Negative | M07 | 1.32 | 1.28 | 1.25 | |
| M08 - 8.5% Sucrose | M08 | 1.32 | 1.33 | 1.33 | |

pH

There were no significant changes in pH at any condition and timepoint for all formulations, as shown in Table 20 below.

TABLE 20
pH

| | | Timepoint | | | | | | |
|---|---|---|---|---|---|---|---|---|
| Formulation | Material | T₀ | T_{3 D} | T_{1 W} | T_{2 W} | T_{3 W} | T_{4 W} | T_{5 W} |

−70° C.

| M05 - Control | M05 | 7.3 | NT | 7.2 | 7.3 |
|---|---|---|---|---|---|
| M06 - Ca Negative | M06 | 7.3 | | 7.3 | 7.3 |
| M07 - Ca/Mg | M07 | 7.3 | | 7.3 | 7.4 |
| Negative | | | | | |
| M08 - 8.5% Sucrose | M08 | 7.2 | | 7.3 | 7.3 |

5° C.

| M05 - Control | M05 | 7.3 | NT | 7.3 | NT | 7.2 | 7.3 |
|---|---|---|---|---|---|---|---|
| M06 - Ca Negative | M06 | 7.3 | | 7.3 | | 7.3 | 7.3 |
| M07 - Ca/Mg | M07 | 7.3 | | 7.2 | | 7.3 | 7.3 |
| Negative | | | | | | | |
| M08 - 8.5% Sucrose | M08 | 7.2 | | 7.2 | | 7.2 | 7.2 |

25° C.

| M05 - Control | M05 | 7.3 | NT | 7.3 | NT | 7.2 | NT |
|---|---|---|---|---|---|---|---|
| M06 - Ca Negative | M06 | 7.3 | | 7.3 | | 7.3 | |
| M07 - Ca/Mg | M07 | 7.3 | | 7.3 | | 7.2 | |
| Negative | | | | | | | |
| M08 - 8.5% Sucrose | M08 | 7.2 | | 7.2 | | 7.2 | |

40° C.

| M05 - Control | M05 | 7.3 | 7.1 | 7.1 | NT |
|---|---|---|---|---|---|
| M06 - Ca Negative | M06 | 7.3 | 7.3 | 7.3 | |
| M07 - Ca/Mg | M07 | 7.3 | 7.3 | 7.3 | |
| Negative | | | | | |
| M08 - 8.5% Sucrose | M08 | 7.2 | 7.1 | 7.2 | |

### Mass Spectrometry Characterization (Deamidation)

Deamidation results as determined by mass spectrometry are presented below in Table 21. Only samples stored at 40° C. were used for this testing as samples stored at lower temperatures are not expected to show significant differences between formulations over the 5-week study period.

Results are presented using the To of the control formulation as the To of all formulations. Among all the deamidation sites, the N57G and N94H hotspots in AAV VP1 are the two which showed the most significant increase in deamidation over 3 weeks at 40° C. Other sites also showed an increase of deamidation, but to a much lesser extent (not shown). These two hotspots are especially important, as they are expected to affect transduction efficiency. The Ca/Mg negative formulation showed the most significant increase in deamidation. The other formulations (M05, M06, and M08) all had similar levels of deamidation increase after 3 days and 3 weeks at 40° C.

TABLE 21
Deamidation by Mass Spectrometry (% at 40° C.)

| | | Timepoint | | | | | |
|---|---|---|---|---|---|---|---|
| | | N57G (VP1) | | | N94H (VP1) | | |
| Formulation | Material | T₀* | T_{3 D} | T_{3 W} | T₀* | T_{3 D} | T_{3 W} |
| M05 - Control | M05 | 7.8 | 14.4 | 41.1 | 5.1 | 6.7 | 30.4 |
| M06 - Ca Negative | M06 | 7.8 | 18.6 | 43.5 | 5.1 | 6.4 | 33.9 |
| M07 - Ca/Mg Negative | M07 | 7.8 | 23.1 | 57.4 | 5.1 | 8.6 | 54.6 |
| M08 - 8.5% Sucrose | M08 | 7.8 | 16.0 | 38.7 | 5.1 | 6.0 | 23.7 |

*T₀ was tested on M05 material and used as T₀ for all formulations.

### Conclusions

Three possible variations on the current SB-525 formulation were evaluated: one with no calcium, one with neither calcium nor magnesium, and one with a higher concentration of sucrose. Examples 1 and 2 show the random generation of very many white-flake particles appearing in both buffer and drug product, especially after freeze/thaw cycling followed by being kept on stability at either 25° C. for longer than 1 week, or 40° C. for 3 days or longer. The particles were identified as calcium phosphate.

The results here demonstrate that calcium chloride is a non-essential excipient for product stability at intended and stressed storage conditions. Additionally, no significant difference was observed for “calcium negative” and “high sucrose” formulations for up to 2 weeks at 40° C., 4 weeks at 25° C., and 5 weeks at 5° C. and −70° C. However, it was observed that removal of both calcium and magnesium from the formulation resulted in changes in drug product quality attributes, including showing larger drops in vg and vp titer as well as higher deamidation and aggregation, especially when kept at 40° C. In addition, it was found that through 5 weeks, only the previous (control) formulation continued to demonstrate the white flake-like particles.

### Example 4: Further Reformulation of SB-525

In order to prevent particle generation, three types of changes to the formulation were tested in this Example: (1) increasing the sucrose concentration from 1% to 5-10% to increase stability against FT stress, (2) removing the divalent cations to remove the source of the particles and prevent potential solubility issues, and (3) increase NaCl to shift the ionic strength of the formulation.

More specifically, the experiments in this Example assessed the stability of SB-525 buffer when reformulated to one of several variants in comparison to the previous (control) formulation; that is, 172 mM NaCl, 8.10 mM Na₂HPO₄, 2.68 mM KCl, 1.47 mM KH₂PO₄, 0.90 mM CaCl₂, 0.49 mM MgCl₂, 1% (w/v) sucrose, and 0.05% (w/v) poloxamer 188, pH 7.0-7.6. Formulations were filled at 5 mL into 6 mL AT vials, subjected to 5 uncontrolled FT cycles (≤−65° C. to ambient), and then placed on thermal stability at 2-8° C. (accelerated; liquid storage) and 25° C. (stressed; liquid storage) to evaluate the various buffers against particle formation.

### Study Design

Formulations tested, as shown in Table 22, were formulated by addition of sodium and potassium chlorides and phosphates to water, followed by addition of sucrose. If present, magnesium chloride, followed by calcium chloride, were then prepared in separate 50 mL solutions, transferred to the larger solution. Poloxamer 188 was then added to the solution. The formulations were then QS'd (quantum satis), pH tested, and filtered over a 0.22 μm PES filter.

TABLE 22
Formulations

| Material | Formulation | Difference from Control |
|---|---|---|
| 0139-M03 | 200 mM NaCl | Increase NaCl to 200 mM |
| 0139-M04 | 5% sucrose | Increase sucrose to 5% |
| 0139-M05 | 8.5% sucrose | Increase sucrose to 8.5% |
| 0139-M06 | 10% sucrose | Increase sucrose to 10% |
| 0139-M07 | No CaCl₂ | Removed CaCl₂ |
| 0139-M08 | No MgCl₂ or CaCl₂ | Removed CaCl₂ and MgCl₂ |
| 0139-M09 | Control | N/A |

With these formulations, the AT vials were filled at 5 mL, subjected to 5 uncontrolled freeze/thaw cycles, and then placed on either 5° C. or 25° C. stability. The stability pull schedule is outlined in Table 23. In the Table, X refers to appearance (liquid); A refers to osmolality (freezing point depression), conductivity, viscosity, and density; B refers to light obscuration with HIAC; C refers to P188 concentration; and D refers to pH.

TABLE 23
Stability Schedule 0139-M03 to -M08

| Temp (° C.) | T₀ | FT1 | FT2 | FT3 | FT4 | FT5* | T_{5 D} | T_{8 D} | T_{2 W} | T_{3 W} | T_{4 W} | T_{6 W} | T_{8 W} |
|---|---|---|---|---|---|---|---|---|---|---|---|---|---|
| 5 (±3) | XABD | X | X | XBD | X | XBD | X | X | XC | XB | XB | XB | XB |
| 25 (±3) | | | | | | | XBC | | X | | | | |

*The 5 FT sample was the T₀ for the thermal stability samples.

### Formulation Characterization

SB-525 buffer formulations were tested to determine conductivity, osmolality, density, and viscosity. P188 concentration was also measured at 2.5 weeks of 5° C. storage, and 5 days of 25° C. The results to these tests are shown in Table 24 below. The results in conductivity, osmolality, density, and viscosity for M03-M09 all follow expected trends—higher amounts of sucrose resulted in a higher osmolality, density, and viscosity; higher NaCl resulted in a higher osmolality and conductivity. Results for the concentration of P188 were within method variability for each set of results, confirming the correct P188 concentration was achieved during formulation.

TABLE 24
Formulation Characterization

| | Conductivity | Osmolality | Density¹ | Viscosity² | P188 (μg/mL) | |
|---|---|---|---|---|---|---|
| Formulation | (ms/cm) | (mOsm/kg) | (g/mL) | (Cp) | T_{2.5 W} 5° C. | T_{5 D} 25° C. |
| M03 - | 20.20 | 433 | 1.011915 | 1.165 | 590 | 484 |
| 200 mM NaCl | | | | | | |
| M04 - | 16.12 | 515 | 1.026105 | 1.285 | 630 | 467 |
| 5% Sucrose | | | | | | |
| M05 - | 15.37 | 647 | 1.039606 | 1.382 | 635 | 486 |
| 8.5% Sucrose | | | | | | |
| M06 - | 15.12 | 704 | 1.045258 | 1.429 | 633 | 457 |
| 10% Sucrose | | | | | | |
| M07 - | 17.91 | 380 | 1.010685 | 1.112 | 663 | 488 |
| Ca Negative | | | | | | |
| M08 - | 17.88 | 379 | 1.010806 | 1.123 | 672 | 471 |
| Ca/Mg Negative | | | | | | |
| M09 - | 17.86 | 384 | 1.010854 | 1.105 | 668 | 475 |
| Control | | | | | | |

¹Temperature during density measurement was recorded as 20.0° C.
²Temperature during viscosity measurement was recorded as 20.0° C. and shear rate was recorded as 2000 s−1.

pH

No significant change in pH was observed for any formulation during FT cycling, as shown below in Table 25.

TABLE 25
pH Results

| | FT Cycle | | |
|---|---|---|---|
| Formulation | T₀ | FT3 | FT5 |
| M03-200 mM NaCl | 7.2 | 7.3 | 7.2 |
| M04-5% Sucrose | 7.2 | 7.2 | 7.2 |
| M05-8.5% Sucrose | 7.2 | 7.2 | 7.2 |
| M06-10% Sucrose | 7.1 | 7.2 | 7.2 |
| M07-Ca Negative | 7.3 | 7.3 | 7.3 |
| M08-Ca and Mg Negative | 7.3 | 7.3 | 7.3 |
| M09-Control | 7.2 | 7.3 | 7.2 |

### Visible Appearance

All samples displayed B9 color and less than or equal to opalescence reference 1 for each condition and timepoint tested. Visible appearance results showed that only the control formulation (M09) consistently displayed too many to count (TMTC), white flake-like particles from 5 days to up to 8 weeks at 5° C. and 25° C. The 5% sucrose formulation exhibited random generation of TMTC visible particles. Many samples also showed 1-5 fibrous particles. While these particles were not identified for this study, fiber-like particles are more characteristic of exogenous particles (e.g., filter particles) versus the flake-like particles that are characteristic of calcium phosphate. Also, appearance of fiber-like/exogenous particles was negligible for lab-based development studies where DP is not manufactured under tightly controlled environmental conditions.

### Sub-Visible Particle Count

Sub-visible particle analysis by the HIAC showed the only formulation with significant and consistent sub-visible particle count was the control formulation (M09), exacerbated by ongoing storage at a higher stability condition (25° C.). Other formulations (M03-M08) showed non-significant particle counts, independent of time or condition, and demonstrated no overall trend.

### Conclusions

This Example evaluated the effects of freeze-thaw stress and thermal stability on several formulation variants from the previous (control) SB-525 formulation. Seven different formulations were filled into AT crystal closed vials, freeze-thaw cycled, and stored at 5° C. and 25° C. for 8 weeks. Across all study conditions, only the control formulation continued to demonstrate the white flake-like particles consistently.

After FT cycling, no formulation exhibited significant visible particles formation or changes in pH, and thus all formulations were placed on short-term stability at 5° C. and 25° C. over a period of 8 weeks. Upon placing on stability, the only formulation shown to consistently generate significant visible particles was the control formulation (M09). Although the particle results were comparable (i.e., no significant visible particle generation), the “200 mM NaCl” (M03), “5% sucrose” (M04), and “10% sucrose” (M06) formulations were not selected for continued reformulation studies. Particularly, the “5% sucrose” formulation (M04) exhibited some formation of white flake-like particles, indicating that this concentration of sucrose may not be sufficient to prevent particle generation due to FT stress, and “10% sucrose” (M06) would introduce manufacturing challenges due to its higher viscosity. Therefore, based on these results, the “8.5% sucrose” (M05), “calcium negative” (M07), and “calcium and magnesium negative’ (M08) formulations appear promising.

### Example 5: Final Formulation for SB-525

The composition and description of SB-525 (PF-07055480) Drug Substance (DS) is shown in Table 26. The formulation had a final pH of 7.3±0.3. Excipient concentration was obtained wholly or in part through the addition of the base buffer (Phosphate Buffered Saline). DS concentration target was 1.0E+13 vg/mL (0.5E+13-2.5E+13 vg/mL), with a DS range of 50-250% of DS target.

TABLE 26
SB-525 Drug Substance Formulation Description

| | | | Concentration | Concentration |
|---|---|---|---|---|
| Raw Material | Chemical Formula | FW | (mM) | (g/L) |
| SB-525 | N/A | N/A | 1.0E+13 vg/mL | 1.0E+13 vg/mL |
| Magnesium Chloride | MgCl₂ · 6H₂O | 203.30 | 0.49 | 0.10 |
| Hexahydrate | | | | |
| Potassium Chloride | KCl | 74.5513 | 2.68 (mM) | 0.20 (g/L) |
| Potassium Phosphate | KH₂PO₄ | 136.086 | 1.47 | 0.20 |
| Monobasic | | | | |
| Sucrose | Cl₂H₂₂O11 | 342.2965 | 1% (w/v) | 10.00 |
| Sodium Chloride | NaCl | 58.44 | 172 | 10.05 |
| Poloxamer 188 (P188) | (C₃H₆O • C₂H₄O)x | 8350 | 0.05% (w/v) | 0.50 |
| Sodium Phosphate Dibasic | Na₂HPO₄ | 141.96 | 8.10 | 1.15 |
| Anhydrous | | | | |
| WFI | H₂O | 18.015 | q.s. to 1 mL | q.s. to 1 mL |

The composition and description of SB-525 (PF-07055480) Drug Product is shown in Table 27. The DP had a final pH of 7.3±0.3. Excipient concentration was obtained wholly or in part through the addition of the base buffer (Phosphate Buffered Saline). DP concentration target was 1.0E+13 vg/mL (0.3E+13-3.0E+13 vg/mL), with a DP range of 30-300% of DP target.

TABLE 27
SB-525 Drug Product Formulation Description

| | | Concentration | Unit Formula | Unit Formula |
|---|---|---|---|---|
| Name of Ingredients | Function | (mM) | (mg/mL) | (mg/vial) |
| SB-525 | Active Ingredient | 1.0E+13 vg/mL | 1.0E+13 vg/mL | 6.0E+13 vg/vial |
| Sodium Phosphate | Buffering | 8.10 | 1.15 | 6.75 |
| Dibasic Anhydrous | Components | | | |
| Potassium Phosphate | | 1.47 | 0.20 | 1.20 |
| Monobasic | | | | |
| Sodium Chloride | Stabilizers | 172 | 10.05 | 60.30 |
| Potassium Chloride | | 2.68 | 0.20 | 1.20 |
| Magnesium Chloride | | 0.49 | 0.10 | 0.60 |
| Hexahydrate | | | | |
| Sucrose | Cryoprotectant | 1% (w/v) | 10.00 | 60.00 |
| Kolliphor (Poloxamer) | Surfactant | 0.05% (w/v) | 0.50 | 3.00 |
| P 188 | | | | |
| WFI | Solvent | q.s. to 1 mL | q.s. to 1 mL | q.s. to nominal |
| | | | | fill volume |

### Container and Fill Volume

Filtered Drug Substance was packaged in sterile high-density polyethylene (HDPE) bottles. The DP was filled into AT 10 mL cyclo-olefin copolymer (COC) vials with in-place thermoplastic elastomer stoppers (Aseptic Technologies VIA-101800). Both DS and DP were stored at −60° C. to −90° C. Each vial contained 6.4 mL of DP (intended extractable volume 6 mL), with 6.4E+13 (nominal 6.0E+13) vg SB-525 AAV.

### Characterization of DP

The viscosity, osmolality, density, and conductivity of the SB-525 DP were measured. The density was 1.0106 g/mL (20° C.). The viscosity was 1.112 cP (20° C.). The osmolality was 374 mOsm/kg. The conductivity was 17.80 mS/cm (20° C.).

### Example 6: AAV6 Viral Vector Drug Product Formulations

Examples 6-8 below describe an additional reformulation and accelerated stability study performed to evaluate AAV2/6 viral vector drug product formulations containing various concentrations of divalent cation salts. As in the previous example, the viral vector has an AAV6 capsid and a recombinant genome containing AAV2 ITRs. The viral vector here carries a transgene that encodes alpha-L-iduronidase (IDUA). This transgene would help patients who is IDUA-deficient, such as patients with mucopolysaccharidosis type I (MPS I), also known as Hurler Syndrome (a lysosomal storage disorder). The AAV genome is shown as sequence No. 28 in Table 5 of US2020/0246486. Because the viral genome is located within the AAV capsid and not exposed to the product formulation, the observations made in Examples 6-8, like those in the previous examples, are expected to be applicable to AAV6 vectors that carry other transgenes.

The study also was performed to identify a new product formulation that does not form precipitates following freeze/thaw cycles. The stability of four AAV6 viral vector drug product formulations containing various concentrations of divalent cation salts was assessed.

The formulations tested contained various concentrations of divalent cation salts (Ca₂₊ and Mg²⁺). As described above, prior AAV6 formulations as well as formulation buffer without AAV6 were all observed to form small amounts of visible precipitates following multiple freeze/thaw cycles. The precipitated particles were determined to be comprised of calcium phosphate salts. Since the drug product will experience freeze/thaw cycles during typical use, the chance of salt precipitation would pose a risk to patient safety as well as a risk to product quality. The study shown in Examples 6-8 below aimed to find improved formulations that do not have such a precipitation issue.

In the study, samples of AAV6 viral vector product were prepared in different product formulations by tangential flow filtration and spiking samples with higher concentration “spike buffers.” The reformulated samples were then incubated at various accelerated stability conditions, and the samples' quality attributes were evaluated following incubation. Quality attribute results were assessed with consideration for known method variability where appropriate. Outliers and trending results over the study duration were identified for each of the four formulations. A scoring of Pass, Neutral, or Fail was assigned for each quality attribute and formulation based on the results at each study endpoint. Finally, the overall quality attribute stability scorings of each four formulations were compared against each other. The study is described in detail below.

### Sample Preparation

AAV6 product formulations prepared herein contained AAV6 donor vector at approximately 1.0E+13 vg/mL suspended in formulation buffer F0 as described in Table 1 below. Buffer F0 is based upon a recipe of Dulbecco's phosphate buffered saline that includes divalent cations calcium (as CaCl₂) and magnesium (as MgCl₂), approximately 35 mM additional sodium chloride, and is formulated to 1% w/v sucrose and 0.05% w/v poloxamer 188.

In order to transfer the AAV6 material into alternate buffer formulations, buffer exchange by tangential flow filtration was performed as shown in FIG. 2. Briefly, several vials of the AAV6 drug product formulated in buffer F0 were thawed and transferred to a single Ultrafiltration/Diafiltration (UF/DF) reservoir. Ultrafiltration was first performed to concentrate the material approximately 2×. Then the concentrated material was diafiltered with at least 10×equivalent volumes of formulation buffer F1. The recovered intermediate material, labeled “A” in FIG. 2, should thus contain approximately 2.0E+13 vg copies/mL suspended in formulation buffer F1.

The intermediate UF/DF product “A” was then separated into three volumes and formulated into three different buffers. Three additional formulation buffers (F1, F2, and F3; Table 28) were derived from Dulbecco's phosphate buffered saline with additional sodium chloride, sucrose, and poloxamer 188 (e.g., Pluronic® F-68). These three buffers contained no calcium and instead had different amounts of magnesium. Buffer F1 contained no calcium or magnesium components, Buffer F2 contained the equivalent molar concentration of magnesium as F0, and Buffer F3 contained additional magnesium accounting for the moles of calcium removed from buffer F0.

TABLE 28
Composition of Formulation Buffers

| | | | F2 | F2 | F3 | F3 |
|---|---|---|---|---|---|---|
| Component (unit) | F0 | F1 | (6x conc) | (1x conc) | (6x conc) | (1x conc) |
| NaCl (mM) | 171.8 | 171.8 | 171.8 | 171.8 | 171.8 | 171.8 |
| KCl (mM) | 2.7 | 2.7 | 2.7 | 2.7 | 2.7 | 2.7 |
| Na₂HPO₄ (mM) | 8.1 | 8.1 | 8.1 | 8.1 | 8.1 | 8.1 |
| KH₂PO₄ (mM) | 1.5 | 1.5 | 1.5 | 1.5 | 1.5 | 1.5 |
| CaCl₂ (mM) | 0.9 | 0.0 | 0.0 | 0.0 | 0.0 | 0.0 |
| MgCl₂ (mM) | 0.5 | 0.0 | 3.0 | 0.5 | 8.4 | 1.4 |
| Sucrose (% w/v) | 1.00% | 1.00% | 1.00% | 1.00% | 1.00% | 1.00% |
| Poloxamer 188 (% w/v) | 0.05% | 0.05% | 0.05% | 0.05% | 0.05% | 0.05% |

| | |
|---|---|
| Target pH | 7.1-7.5 |

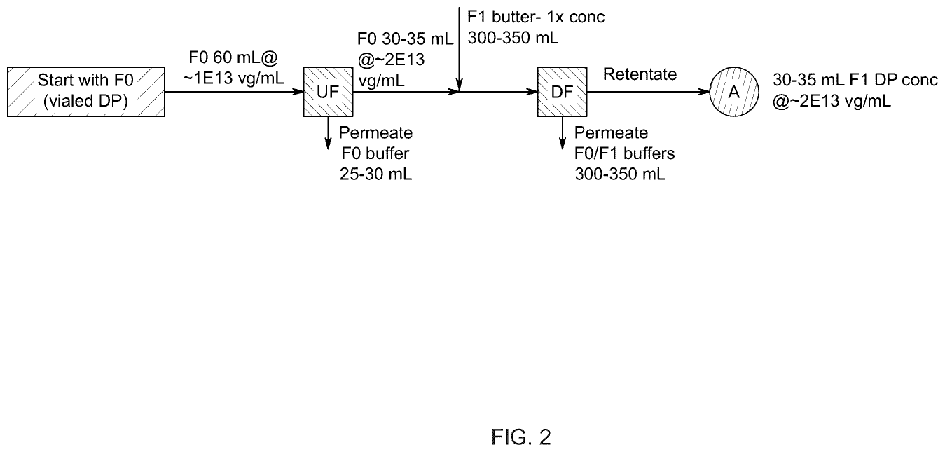

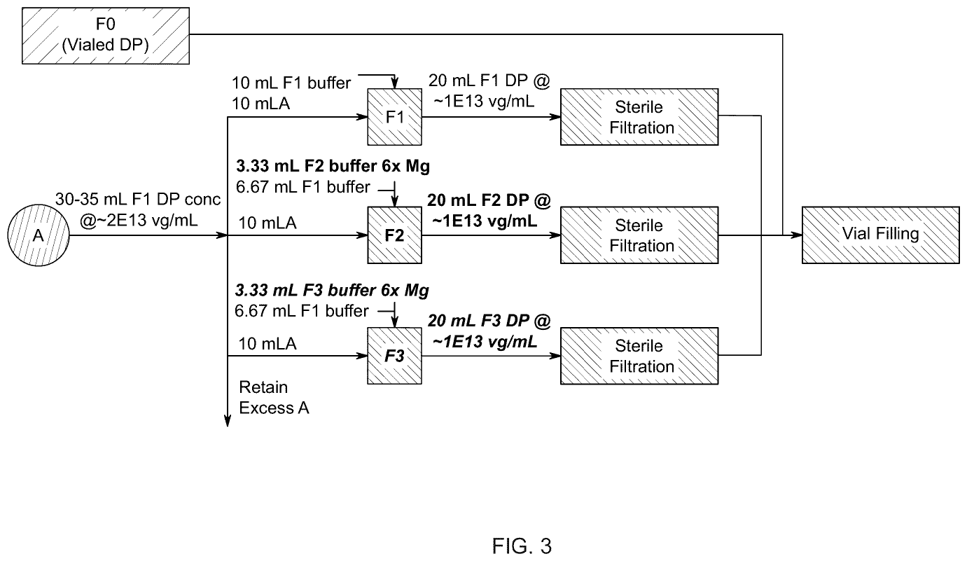

To simplify sample preparation and to allow preparation of different buffers, 6-times higher concentrated “spike buffers” of buffers F2 and F3 were prepared. As shown in FIG. 3, these F2 and F3 “6× conc” buffers when mixed at a 1:5 ratio with F1 yielded the intended composition of buffers F2 and F3, respectively.

To prepare these formulation buffers, individual dry components were weighed off using a microbalance. The components were mixed and dissolved with deionized water for injection. These buffers were then titrated with 0.1 M NaOH or 0.1 M HCI to the intended pH range if needed. Finally, these buffers were formulated with sucrose and poloxamer 188 to the desired concentrations.

The compositions of the prepared drug product formulations were measured with several semiquantitative assays as a check, the results of which are reported in Table 29. Off-the-shelf colorimetric assays were used to measure concentrations of Ca²⁺ (Bio Vision Cat #K380-250) and Mg²⁺ (Bio Vision Cat #K385-1 00). For both colorimetric assays, the sample concentrations were within the assays' dynamic linear ranges, and the reported signals and calculated concentrations trended well with the theoretical sample compositions. Critically, the calcium signals for formulations F1, F2, and F3 all matched the assay negative controls. Thus, these samples contained no calcium, and the TFF sample preparation step was effective at removing any calcium that was present in the F0 starting material. The magnesium assay also confirmed Mg²⁺ was also removed by TFF and spiked into the appropriate concentrations in F2 and F3.

TABLE 29
Experimentally Measured Parameters of Formulated Drug Products

| | | | F2 | F3 |
|---|---|---|---|---|
| Component (unit) | F0 | F1 | (1x conc) | (1x conc) |
| CaCl₂ (mM, by | 1.02 | 0.07 | 0.09 | 0.1 |
| colorimetric assay) | | | | |
| MgCl₂ (mM, by | 0.29 | −0.04 | 0.38 | 0.9 |
| colorimetric assay) | | | | |
| VG titer (vg/ml, by qPCR) | 1.54E+13 | 1.77E+13 | 1.91E+13 | 1.87 E+13 |
| Particle Concentration | 7.79E+12 | 6.38E+12 | 7.30E+12 | 8.23E+12 |
| (p/mL, by MADLS) | | | | |

*MADLS: Multi-Angle Dynamic Light Scattering.

qPCR was also performed to measure vg titer, and multi-angle dynamic light scattering (MADLS) was performed to measure particle concentration. Again, these results were in the expected ranges from sample preparation, indicating that the buffer exchange and formulation steps performed as intended.

The bulk formulated products were sterile filtered and filled at 0.5 mL/vial into 2 mL cyclic olefin polymer (COP) vials (West Pharma/Daikyo CZ Cat #19550057).

### Sample Incubation

Vialed drug products were incubated at various conditions described below. Freeze/thaw cycling was performed with pull dates listed in Table 30.

TABLE 30
Freeze/Thaw Cycling Sample Pull Dates

| | Start | 3X | 5X | 10X |
|---|---|---|---|---|
| Condition | Date | Cycles | Cycles | Cycles |
| Freeze/Thaw cycling −80° C./Ambient | D0 | D2 | D4 | D11 |

Samples were subject to “accelerated, stressed” conditions (40° C./75% relative humidity (RH)) or to “accelerated, ambient” conditions (25° C./60% RH), with pull dates at D3, D7, 2 weeks, 1 month, 2 months, 3 months, or 6 months. Samples were stored at 2-8° C. and analyzed within 24 hours of their pull time.

All pulled samples were aliquoted into polycarbonate flip-top centrifuge tubes for the intended sample testing. Aliquots were stored at 2-8° C. during active testing for no longer than 2 months. If testing was not performed within a reasonable time from the pull date, the aliquots were frozen at ≤−65° C. until testing could occur. All retain samples and remaining materials following testing were frozen at ≤−65° C. for long term storage.

### Example 7: Testing of AAV6 Drug Product Formulations

Sample testing performed included the quality attributes listed in Table 31. Abbreviations are shown as below: DLS, dynamic light scattering; MADLS, multi-angle dynamic light scattering; HMWS, high molecule weight species; SE-LC, size exclusion liquid chromatography; and AEX-LC, anion exchange liquid chromatography. Appearance of the solutions was assessed to confirm that they were clear, colorless, and free of particulates. A solution that was hazy, cloudy, or containing visible particles did not meet the criteria.

TABLE 31
Product Quality Attributes Evaluated

| Category | Quality Attribute | Testing Methods |
|---|---|---|
| General | Appearance, Liquid | Visual inspection |
| | pH | Potentiometric |
| Purity | Monomer Peak Mean Size | DLS |
| | Polydispersity Index | DLS |
| | Monomer Fraction | DLS |
| | HMWS Fraction | DLS |
| | HMWS Fraction | SE-LC |
| | % Full Capsids | AEX-LC |
| Strength | Vector Genome Titer | qPCR |
| | Infectious Titer | TCID₅₀ |
| | VG/TCID₅₀ Ratio | Calculation |
| | Capsid Concentration | ELISA |
| | Monomer Concentration | SE-LC |
| | Monomer Concentration | MADLS |

### Initial Samples

Quality attribute results from all initial formulated drug product samples are shown in Table 32 and Table 33. Results are grouped by the quality attribute categories of general, purity, and strength.

TABLE 32
General and Purity Quality Attribute Results of Initial (t₀) Samples

| Formulation | F0 | F1 | F2 | F3 |
|---|---|---|---|---|
| Appearance (Pass/Fail) | Pass | Pass | Pass | Pass |
| pH | 7.3 | 7.3 | 7.2 | 7.2 |
| Monomer Peak Mean Size (nm) | 29.7 | 33.0 | 33.6 | 33.4 |
| Polydispersity Index | 0.07 | 0.12 | 0.14 | 0.12 |
| % Monomer, DLS | 100.0% | 100.0% | 100.0% | 100.0% |
| % HMWS, DLS | 0.00% | 0.00% | 0.00% | 0.00% |
| % HMWS, SE-LC | 0.2% | 0.5% | 0.5% | 0.4% |
| % Full Capsids | 89.2% | 89.1% | 89.3% | 89.1% |

TABLE 33
Strength Quality Attribute Results of Initial (t₀) Samples

| Formulation | F0 | F1 | F2 | F3 |
|---|---|---|---|---|
| VG Titer (vg/mL) | 7.9E+12 | 8.3E+12 | 9.4E+12 | 7.7E+12 |
| Infectious Titer (TCID₅₀/mL) | 9.3E+09 | 9.3E+09 | 1.1E+10 | 1.1E+09 |
| VG/TCID₅₀ | 1200 | 1100 | 1100 | 8400 |
| Capsid Titer (capsids/mL) | 5.0E+12 | 5.5E+12 | 5.2E+12 | 6.7E+12 |
| Monomer Conc, SE-LC | 9.9E+12 | 9.5E+12 | 9.9E+12 | 9.7E+12 |
| (particles/mL) | | | | |
| Monomer Conc, MADLS | 7.8E+12 | 6.4E+12 | 7.3E+12 | 8.2E+12 |
| (particles/mL) | | | | |

The initial infectious titer result was usually low for F3 when compared against the other initial samples. This also resulted in a much higher vg/TCID ratio than the other samples. This was attributed primarily to the variability of the TCID₅₀ assay. None of the other results appeared to be substantially affected.

### Freeze/Thaw Cycled Samples

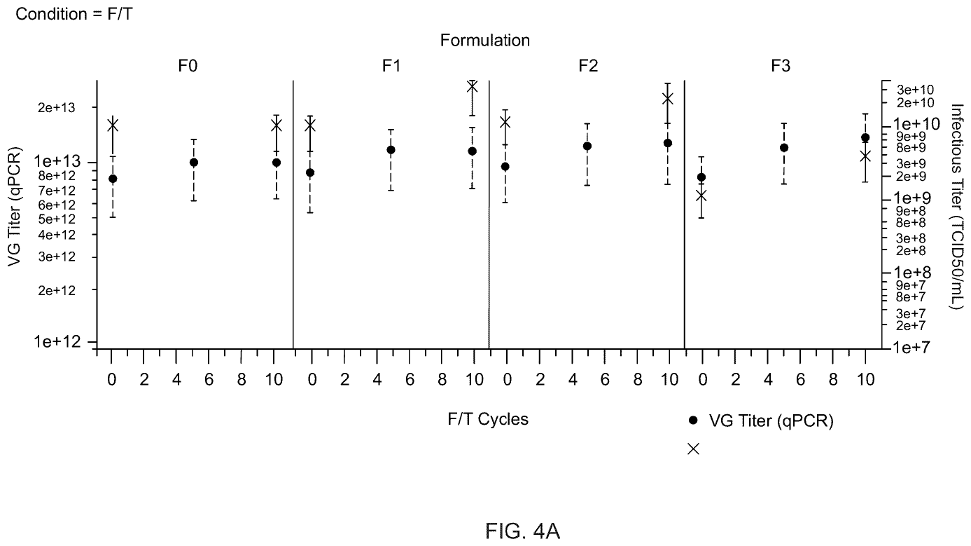

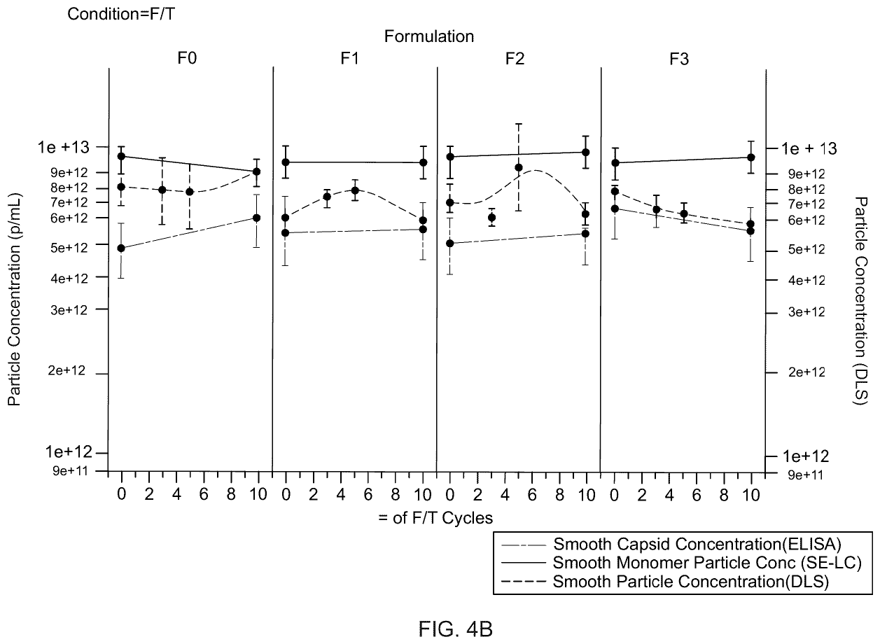

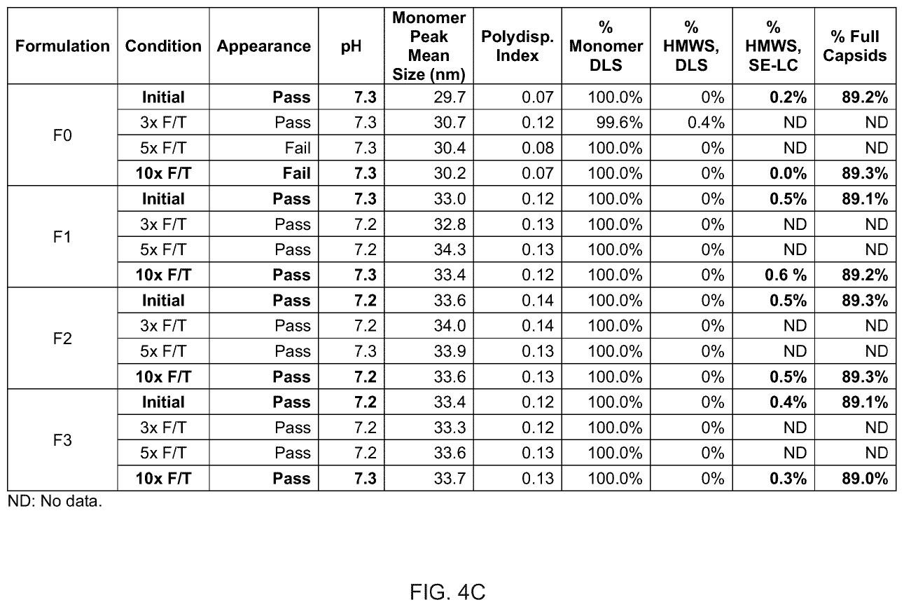

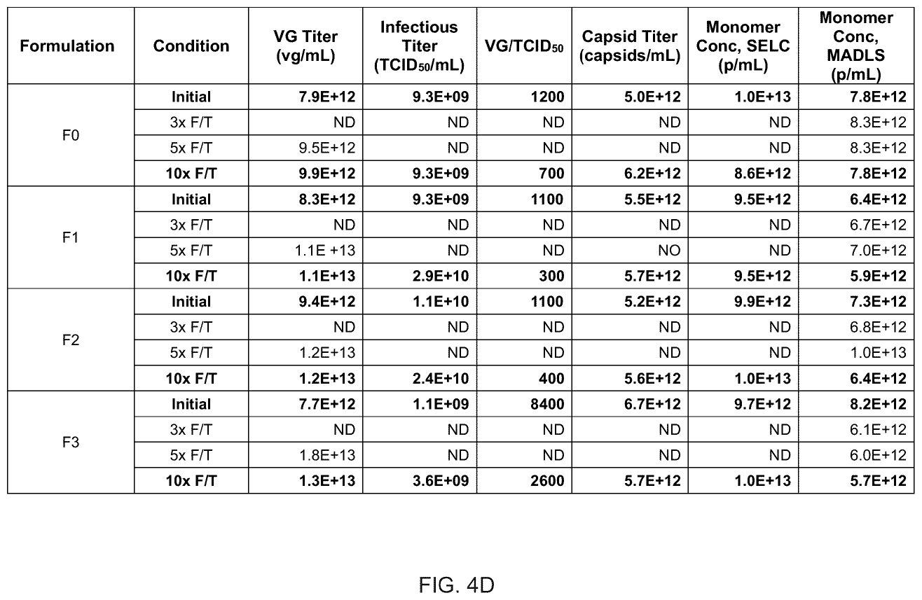

Test results from freeze/thaw cycling of the four different formulations are shown in FIGS. 4A-D. The data show that the freeze/thaw cycling did not affect solution pH, monomer capsid size, sample size distribution, or full capsid content; nor did it generate significant amounts of HMWS or soluble aggregates in all four formulations. Freeze/thaw cycling did, however, lead to visible appearance failures in the initial F0 control formulations. Following 5×F/T cycles, small white flecks of precipitates could be observed, and these were characteristic of the calcium phosphate precipitates previously observed. These precipitates were not detected in formulations F1, F2, or F3. The data also show that freeze/thaw cycling also did not appear to significantly affect any product strength attributes when the variability of test methods is also considered.

### 25° C./60% RH (Ambient) Accelerated Samples

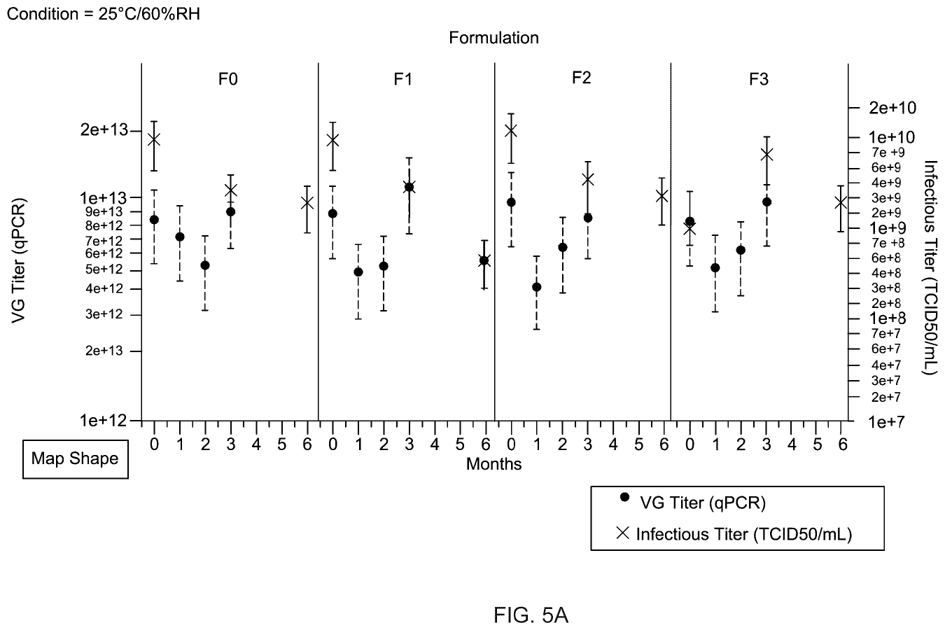

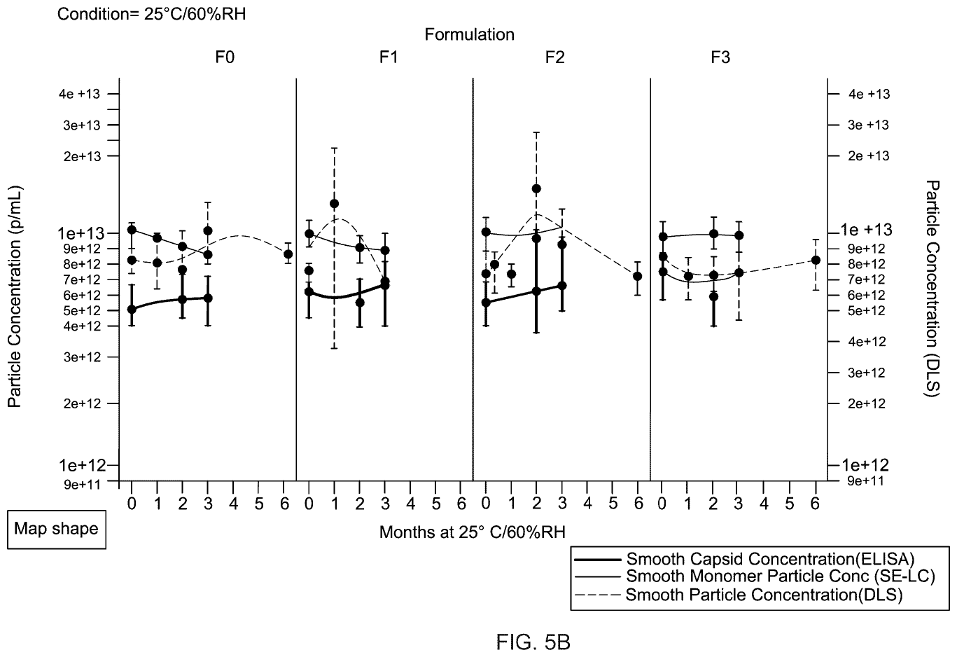

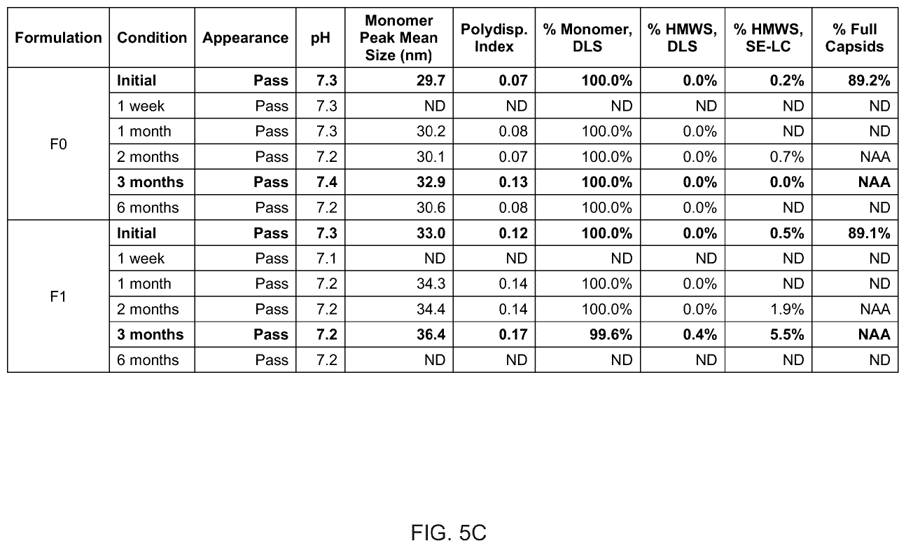

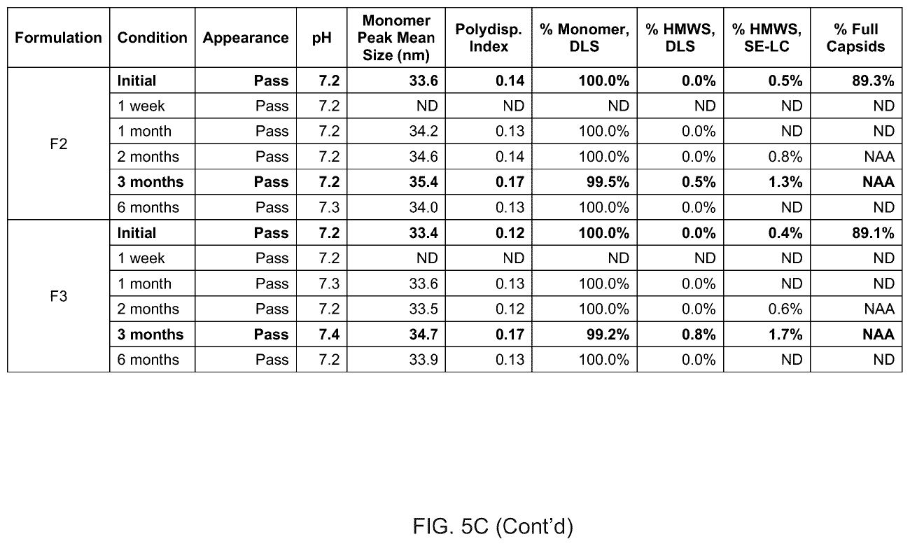

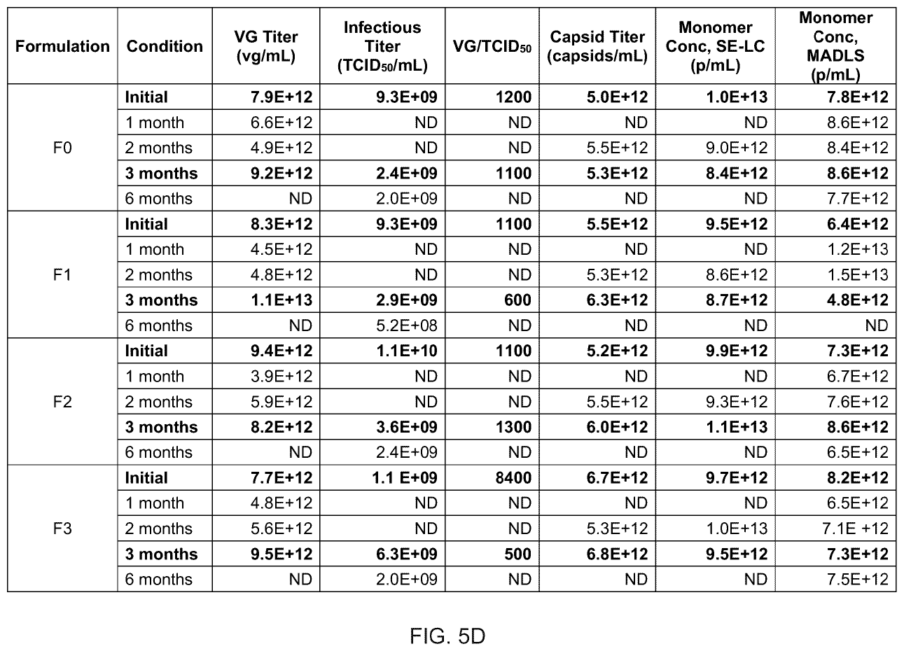

Test results from ambient condition (25° C./60% RH) incubation of the four different formulations are shown in FIGS. 5A-D. The data show that appearance and pH were not affected by the 25° C. condition. The sample size distribution appeared to broaden, increasing the polydispersity index (PDI), and the monomer peak mean size grew slightly as the incubation time increased. High weight molecular species (HMWS) also began to form over incubation time. In general, these HMWS were detected more readily by SE-LC than by DLS. Charge separation of full and empty capsids appears to have been lost following 1-2 months of incubation at 25° C., thus the % full capsids could not be measured by AEX-LC. Capsid titer and monomer concentration were not affected by the 25° C. condition, nor did the vg titer appear to be affected. TCID₅₀ decreased at the 3-month and 6-month time point for F0, F1, and F2 but the same result was not observed with F3.

### 40° C./75% RH Stressed Samples

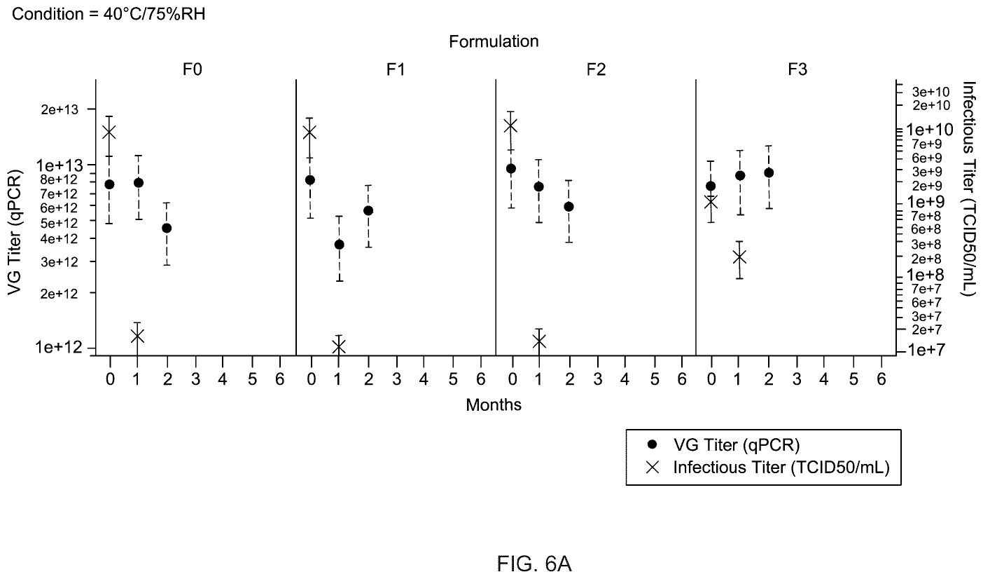

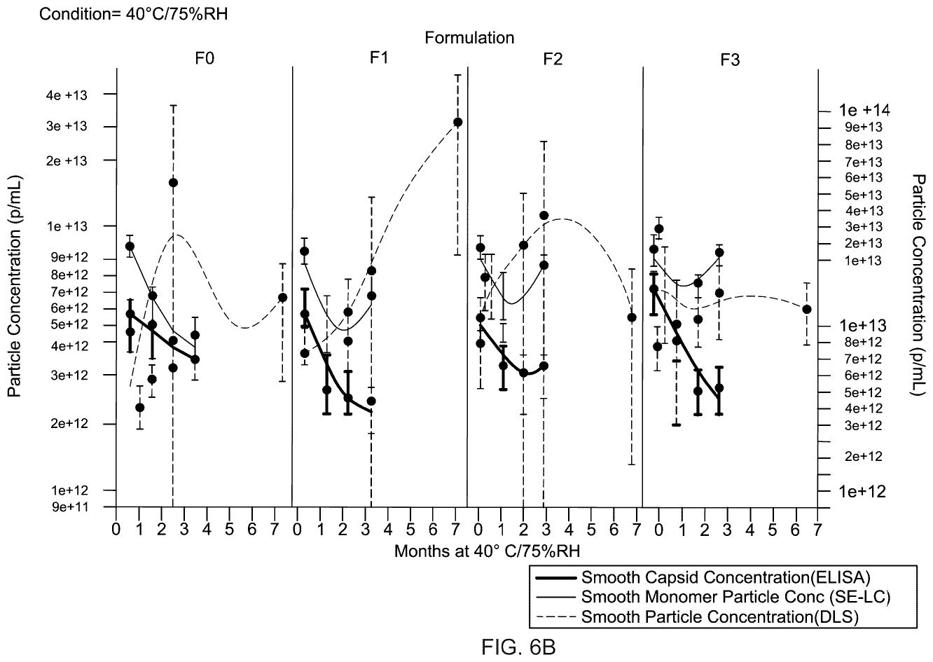

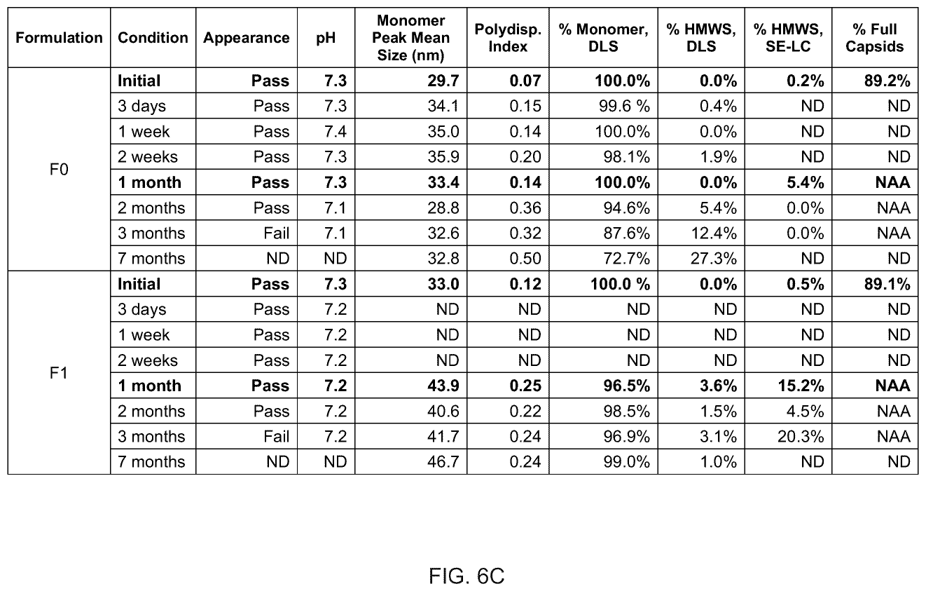

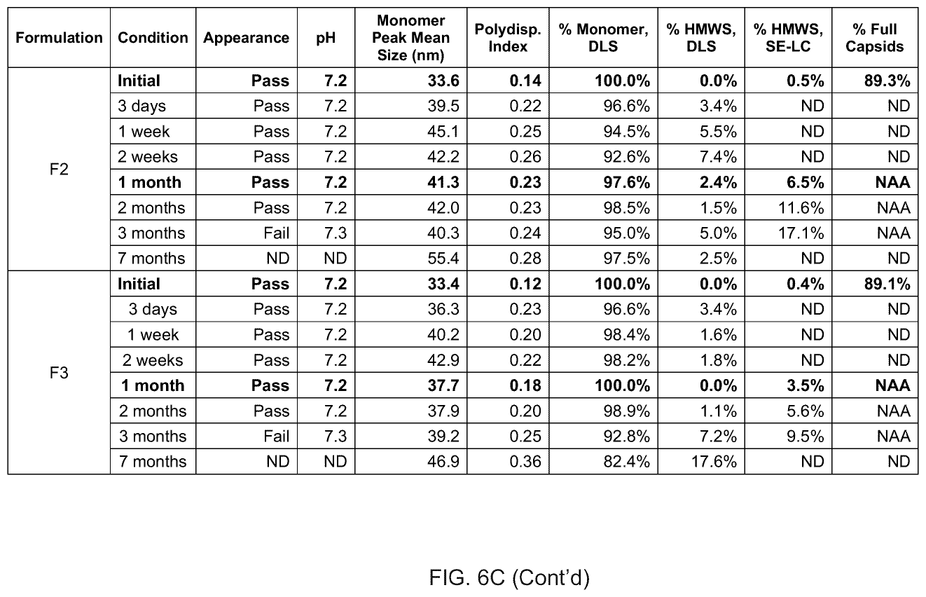

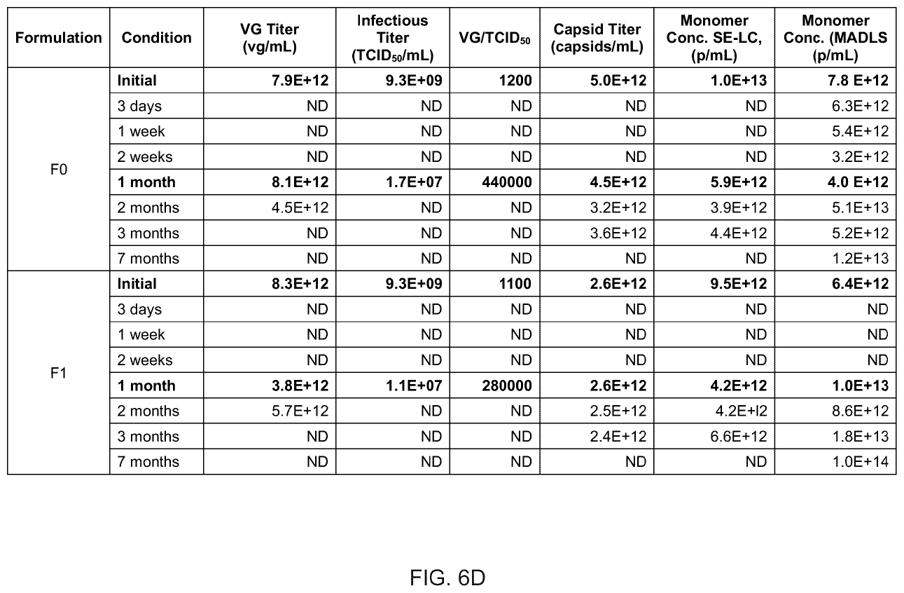

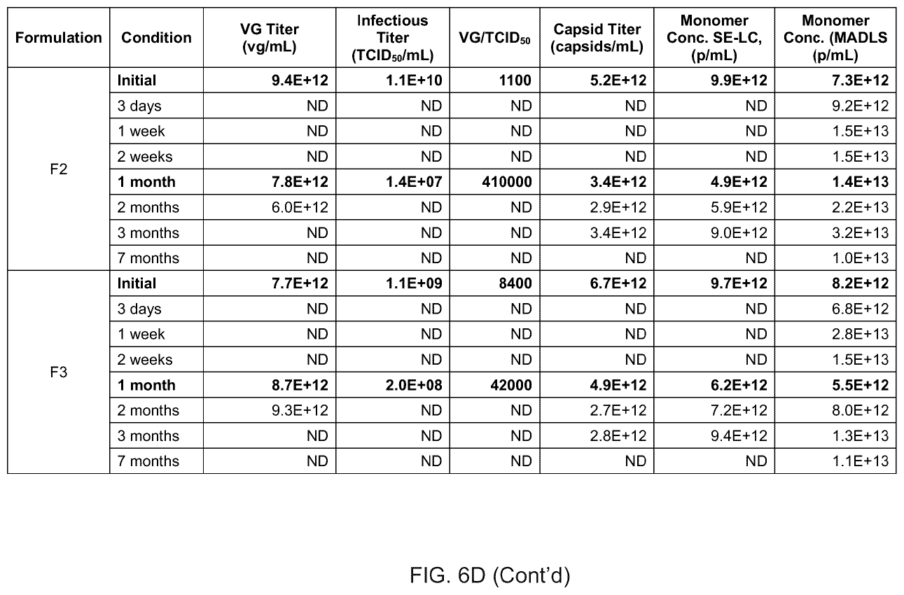

Test results from stressed condition (40° C./75% RH) incubation of the four different formulations are shown in FIGS. 6A-D. The data show that the 40° C./75% RH condition had a much greater impact on several product quality attributes. The particle size distribution broadened substantially and HMWS were forming. SE-LC appeared to be more sensitive to detecting smaller amounts of HMWS than DLS, but when large amounts of HMWS (and possibly larger molecular weight species) were present, these particles might not have been detected by SE-LC. It is likely that large molecular weight species were being filtered or not passing through the LC column to reach the detector in these cases.

Following 3 months at 40° C., all solutions began to appear hazy, possibly due to the formation of large insoluble aggregates or HMWS. The solution pH did not appear to be affected. TCID₅₀ did decrease substantially in all samples after 1 month at 40° C./75% RH with the smallest reduction from initial seen in F3.

### Example 8: Study Endpoint Assessment

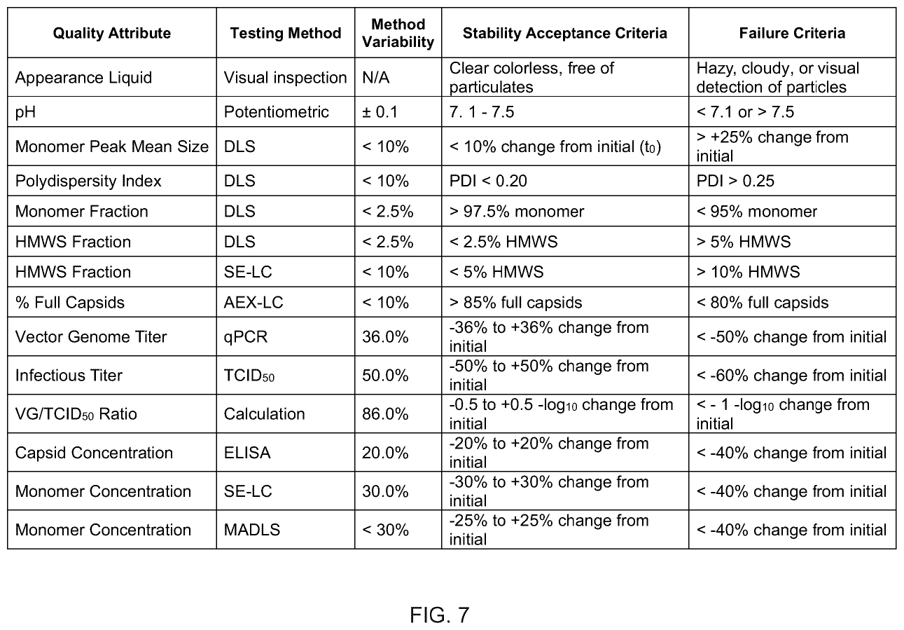

An intra-sample analysis was performed for each formulation. Within each formulation, each study endpoint result for each quality attribute was scored against the acceptance and failure criteria as described in FIG. 7. The intended stability study endpoints were as follows: 10×freeze/thaw cycles of no less than 12 hours at ≤−65° C. and no less than 6 hours at ambient temperature per cycle, 3 months at 25° C./60% RH, and 1 month at 40° C./75% RH. At each study endpoint, the quality attributes as listed in FIG. 7 were determined based on known and established method variability as well as a holistic assessment of all data generated in this study.

To perform the inter-sample analysis or a relative assessment of each formulation, each endpoint score for each quality attribute was compared by formulation. When considering a single quality attribute, an overall Pass, Neutral, or Fail scoring was assigned for each formulation by holistically comparing the relative endpoint scorings across the four considered formulations. For example, if all endpoint results had passed acceptance criteria for a given formulation, an overall Pass score was assigned for that quality attribute for that formulation. The number of neutral and failing endpoint conditions were considered when assigning an overall quality attribute scoring for that formulation. For certain quality attributes such as appearance, one failing endpoint score was enough to score an overall Fail for that formulation. For other quality attributes such as monomer concentration, one failing endpoint at the 40° C. condition was encountered for all formulations (except F3); thus, this failing endpoint was not scored as severely. TCID₅₀ results were included for information only and a scoring was not assigned.

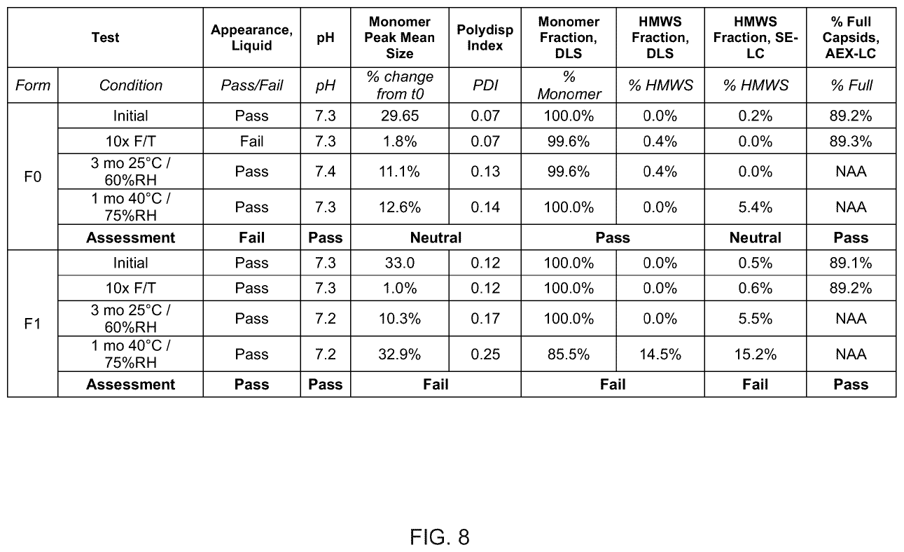

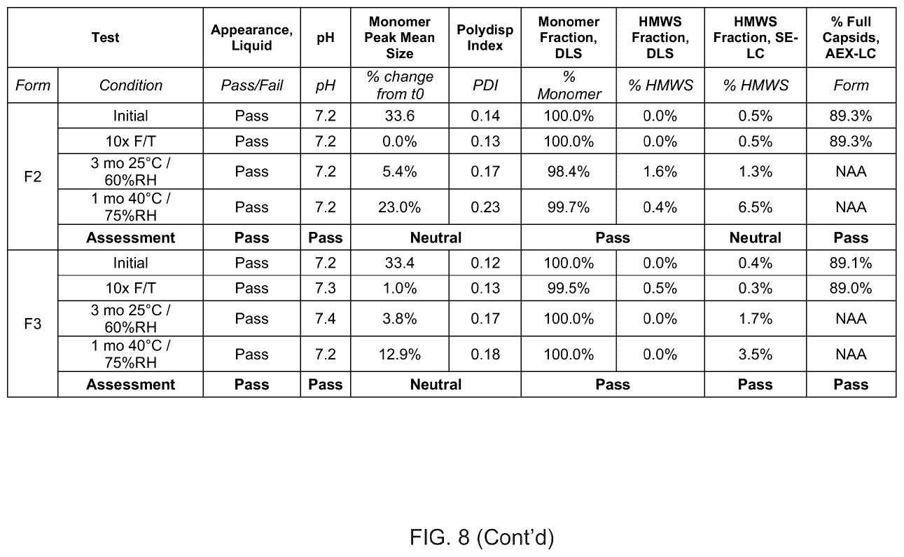

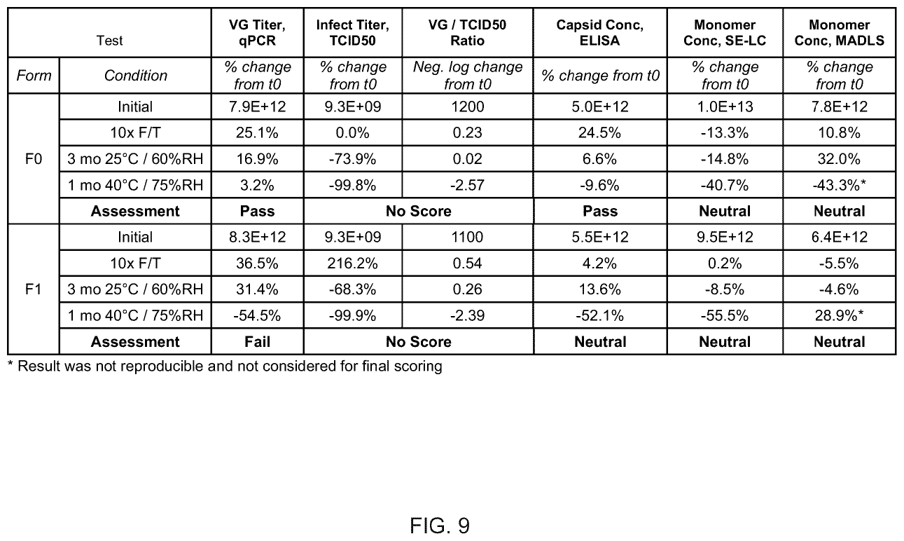

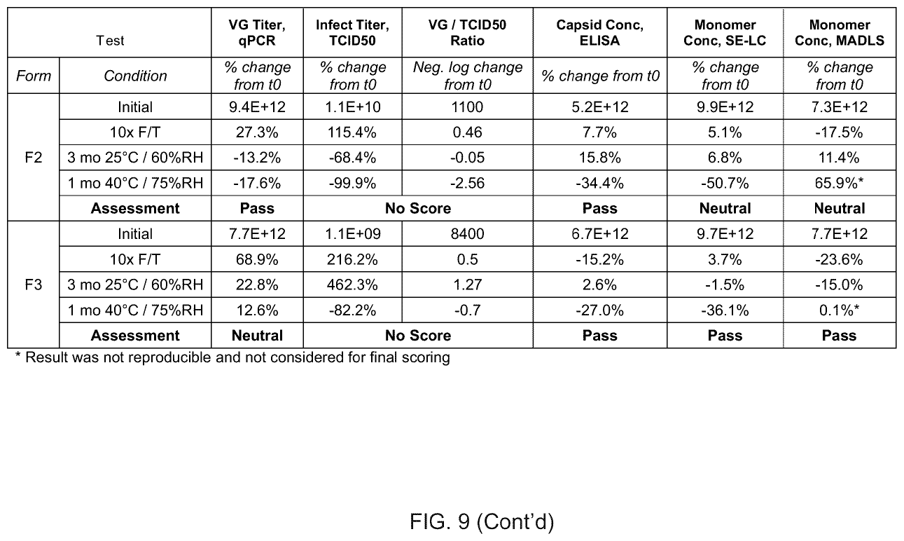

These intra-sample analysis scores are shown in FIG. 8 and FIG. 9. A summary table of all overall quality attribute scores for each formulation is shown in Table 34, where formulation F0 is used as a control.

TABLE 34
Inter-Sample Formulation Assessment Summary

| Test (method) | F0 | F1 | F2 | F3 |
|---|---|---|---|---|
| Appearance, Liquid | Fail | Pass | Pass | Pass |
| (Visual Inspect) | | | | |
| pH (Potentiometric) | Pass | Pass | Pass | Pass |
| Size Distribution (DLS) | Neutral | Fail | Neutral | Neutral |
| HMWS Fraction (DLS) | Pass | Fail | Pass | Pass |
| HMWS Fraction (SE-LC) | Neutral | Fail | Neutral | Pass |
| % Full Capsids (AEX-LC) | Pass | Pass | Pass | Pass |
| VG Titer (qPCR) | Pass | Fail | Pass | Neutral |
| Capsid Conc. (ELISA) | Pass | Neutral | Pass | Pass |
| Monomer Conc (SE-LC) | Neutral | Neutral | Neutral | Pass |
| Monomer Conc. (MADLS) | Neutral | Neutral | Neutral | Pass |

The overall quality attribute scorings over the duration of the accelerated stability study demonstrate that Formulations F3 and F2 are superior to F0 and F1. Formulation F3 appears to have performed the best, with eight overall passing quality attribute scores and only two neutral scores. Formulation F2 also performed well. F0 had an overall failing score for appearance, confirming the initial goal for improving this product formulation. F1 appeared to have the greatest amount of aggregation with the greatest amount of HMWS detected.

### Example 9: Extended Temperature Tests

Stability of SB-525 (PF-07055480) drug product provided at 1.00E+13 vg/mL in formulation buffer was tested at extended temperatures. The formulation buffer contained the following ingredients: 0.49 mM MgCl₂, 2.68 mM KC₁, 1.47 mM KH₂PO₄, 172 mM NaCl, 8.10 mM Na₂HPO₄, 1% (w/v) sucrose, 0.05% (w/v) poloxamer 188, pH 7.4.

Vials containing the drug product were stored at (i) −70° C. for 0 days (T₀), (ii) at −150° C. for 3 days (T_{3-Days}), and (iii) at −150° C. for 14 days (T_{14-Days}). Drug product samples were then analyzed for (i) appearance (liquid), (ii) reduced CE-SDS, (iii) SEC titer_260/280, and (iv) in vitro FVIII activity (bioassay). The formulation buffer sample was also analyzed for (i) container closure integrity.

As shown in Table 35 below, there was no significant change in color, clarity, reduced CE-SDS, SEC titer_260/280 (a surrogate measure for empty:full capsid ratio), or biological activity (% relative potency) when the samples were stored at the tested conditions including storage at −150° C. for up to fourteen days. No impact on container closure integrity was observed after fourteen days storage at −150° C.

TABLE 35
Stability Results

| Test | | T₀ | T_{3-Days} | T_{14-Days} |
|---|---|---|---|---|
| Appearance, | Color | <= B9 | <= B9 | <= B9 |
| Liquid | Clarity | <= Ref. I | <= Ref. I | <= Ref. I |
| | Visible Particles | EFVP | EFVP | EFVP |

| | | | |
|---|---|---|---|
| Container Closure Integrity | N.T. | N.T. | Pass |
| Capsid Protein Purity (CE): % | 95.0 | 94.5 | 94.9 |
| Capsid | | | |
| Capsid Protein Purity (CE): % | 5.0 | 5.5 | 5.1 |
| Other | | | |
| Size Distribution | 98.8 | 99.0 | 99.5 |
| (SEC): % Monomer | | | |
| Size Distribution | 1.2 | NMT 1.0 | NMT 1.0 |
| (SEC): % Aggregates | | | |
| Particle Content | 1.34 | 1.34 | 1.33 |
| (SEC): 260/280 Ratio | | | |
| in vitro FVIII Activity/ | 67% | 69% | 70% |
| Functional Bioassay (% Relative | | | |
| Potency) | | | |

EFVP: Essentially Free of Visible Particles
TMTC: Too many to Count
N.T.: Not tested

### Example 10: Shipping Simulation and Extended Temperature Tests

Stability of the SB-525 drug product under simulated shipping conditions, such as shock, pressure, drop, and vibration, was tested. The SB-525 drug product was provided at 1.00E+13 vg/mL in formulation buffer containing the following ingredients: 0.49 mM MgCl₂, 2.68 mM KCl, 1.47 mM KH₂PO₄, 172 mM NaCl, 8.10 mM Na₂HPO₄, 1% (w/v) sucrose, 0.05% (w/v) poloxamer 188, pH 7.4.

Test sample vials containing the drug product underwent concurrently applied transport hazards (e.g., shock, pressure, drop, and vibration), utilizing a worst-case global transportation profile. Control sample vials were not subjected to those concurrently applied transport hazards. As part of the simulation, test vials were held for 40 hours at −35° C. and subsequently for another 40 hours at −70° C. After simulation, control and test drug product vials were stored at −70° C. and tested at 0 days (T₀), 6 months (T_{6-Months}), and 10 months (T_{10-Months}). Formulation buffer vials were stored at −70° C. and tested at T₀. Drug product samples were analyzed for (i) appearance (liquid), (ii) reduced CE-SDS, (iii) SEC titer_260/280, and (iv) in vitro FVIII activity (bioassay). Formulation buffer samples were analyzed for (i) container closure integrity.

As shown in Table 36 below, there was no significant change in color, clarity, reduced CE-SDS, SEC titer_260/280 (a surrogate measure for empty:full capsid ratio), or biological activity (% relative potency) when the samples were stored at the tested conditions for up to ten months. There was no impact on container closure integrity at T₀.

TABLE 36
Stability Results

| Test | | Sample | T₀ | T_{6-Months} | T_{10-Months} |
|---|---|---|---|---|---|
| Appearance, | Color | Control | <= B9 | <= B9 | <= B9 |
| Liquid | | Test | <= B9 | <= B9 | <= B9 |
| | Clarity | Control | <= Ref. I | <= Ref. I | <= Ref. I |
| | | Test | <= Ref. I | <= Ref. I | <= Ref. I |
| | Visible | Control | EFVP | EFVP | EFVP |
| | Particles | Test | EFVP | EFVP | EFVP |

| | | | | |
|---|---|---|---|---|
| Container Closure Integrity | Control | Pass | N.T. | N.T. |
| | Test | Pass | N.T. | N.T. |
| Genome Titer (qPCR) | Control | 1.11E+13 | 1.02E+13 | 7.96E+12 |
| | Test | 9.07E+12 | 9.29E+12 | 7.81E+12 |
| Capsid Titer (ELISA) | Control | 1.71E+13 | 1.28E+13 | 1.30E+13 |
| | Test | 1.69E+13 | 1.19E+13 | 1.34E+13 |
| Capsid Protein Purity (CE): | Control | 94.0 | 94.3 | 95.0 |
| % Capsid | Test | 94.5 | 94.6 | 94.7 |
| Capsid Protein Purity (CE): | Control | 6.0 | 5.7 | 5.0 |
| % Other | Test | 5.5 | 5.4 | 5.3 |
| Size Distribution | Control | 99.2 | 99.1 | 99.3 |
| (SEC): % Monomer | Test | 99.3 | 98.8 | 99.4 |
| Size Distribution | Control | NMT 1.0 | NMT 1.0 | NMT 1.0 |
| (SEC): % Aggregates | Test | NMT 1.0 | 1.2 | NMT 1.0 |
| Particle Content | Control | 1.33 | 1.32 | 1.34 |
| (SEC): 260/280 Ratio | Test | 1.33 | 1.32 | 1.33 |
| in vitro FVIII Activity/ | Control | 72 | 58 | 62 |
| Functional Bioassay (% | Test | 63 | 58 | 62 |
| Relative Potency) | | | | |

### Example 11: Drug Product Stability Data—24 Months

The purpose of this study was to establish long term (24 months) stability of the SB-525 drug product at the intended storage temperature (−70° C.). For this study, the SB-525 drug product (DP) was purified from Sf9 insect cells and formulated at a target of 1.00E+13 vector vg/mL in 8.10 mM Na₂HPO₄, 1.47 mM KH₂PO₄, 0.49 mM MgCl₂, 2.68 mM KCl, 172 mM NaCl, 1% (w/v) sucrose, and 0.05% (w/v) poloxamer P188, pH 7.3 ±0.3. The DP was then filled at 6.4 mL into 10 mL Aseptic Technologies (AT) crystal closed vials, and stored at −60° C. to −90° C.

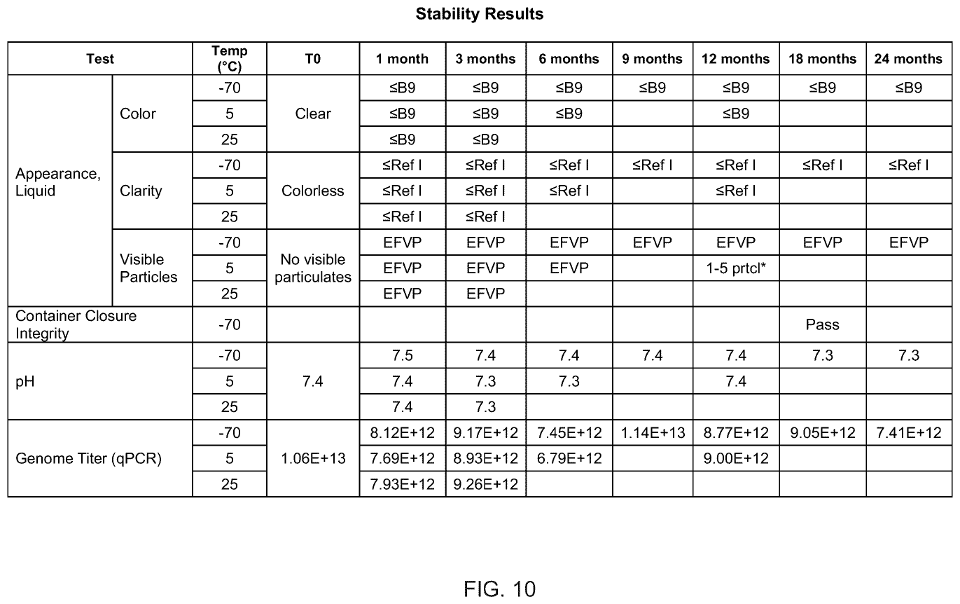

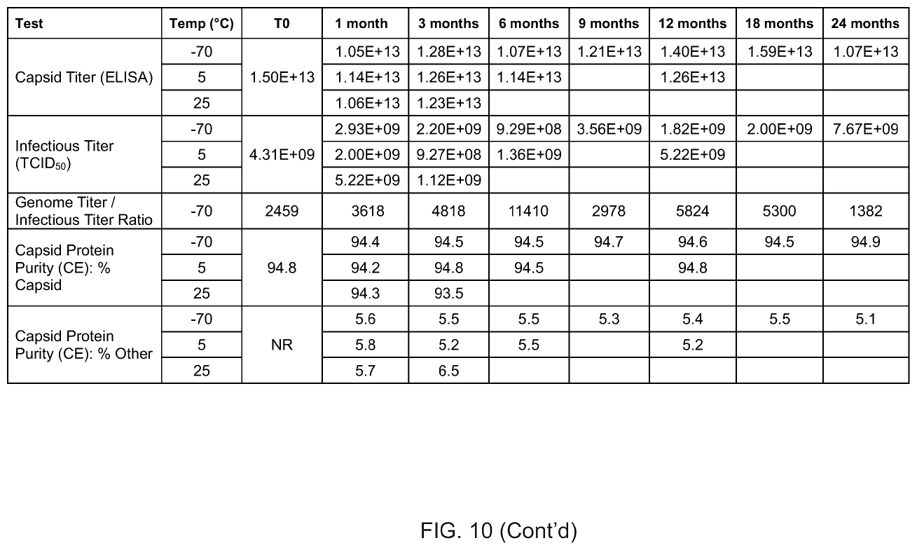

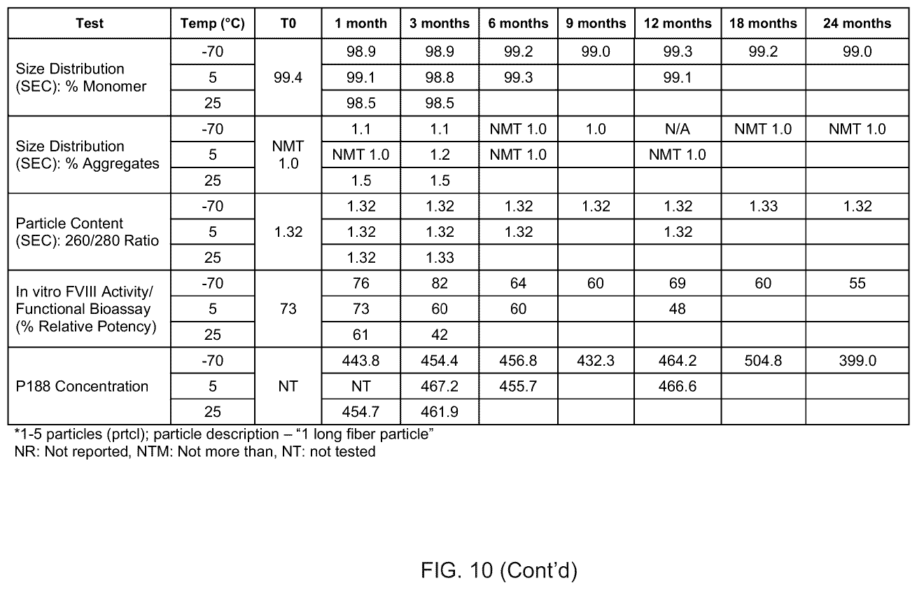

Results from the DP stability study, including 5 cycles of uncontrolled F/T and 24-hour agitation, are shown in FIG. 10. The data show that there was no apparent trend observed in color, clarity, pH, subvisible cumulative particulate matter, capsid purity and VP ratio by R-CGE, % HMMS and UV 260/280 by SEC-HPLC, and capsid titer up to 24 months at −70° C., 12 months at 5° C., and 3 months at 25° C./60% RH. The 12 month, 5° C. sample reported “1 long fiber particle” in solution which appeared to be extrinsic in nature based on morphology and testing in uncontrolled environmental conditions (i.e., open lab space); all other samples reported “essentially free of visible particles.” No change in these quality attributes were observed after five cycles of uncontrolled F/T or 24-hour agitation.

For all the time points and conditions tested, the variability in the results for genome titer and infectious virus titer as well as infectivity ratio were within expected assay variability and did not demonstrate an apparent trend. The results for in vitro relative potency, a sensitive and precise stability-indicating method, show a slight downward trend in potency at the intended storage condition (−70° C.) after 24 months, but the results are within the clinical stability acceptance criterion. A stability-indicating downward trend in potency at 3 months at 25° C./60% RH and at 12 months at 5° C. was also observed.

Additionally, Container Closure Integrity (CCIT) was tested at 18 months at the intended storage condition (−70° C.) by a validated headspace analyzer and recorded a passing result. Six contingency vials were used for this analysis. P188 concentration was also stable for up to 18 months at −70° C., 12 months at 5° C., and 3 months at 25° C./60% RH. A slight decrease in P188 concentration was observed at 24 months at −70° C.

1. A pharmaceutical composition comprising
a recombinant adeno-associated virus (rAAV) vector,
sodium chloride (NaCl)
potassium chloride (KCl),
disodium phosphate (Na₂HPO₄),
monopotassium phosphate (KH₂PO₄),
magnesium chloride (MgCl₂),
a polyol, and
a poloxamer,
optionally wherein the composition comprises no more than about 0.1 mM calcium chloride and has a pH of about 7.1 to about 7.5.

2. The composition of claim 1, wherein the polyol is sucrose.

3. The composition of claim 1 or 2, wherein the poloxamer is poloxamer 188.

4. The composition of any one of claims 1-3, wherein the composition contains about 0.1 to about 2.0 mM magnesium chloride.

5. The composition of claim 4, wherein the composition contains about 0.5 mM or higher magnesium chloride.

6. The composition of claim 4, wherein the composition contains about 1.4 mM magnesium chloride.

7. The composition of claim 4, wherein the composition contains more than about 1.3 mM magnesium chloride.

8. The composition of any one of claims 1-7, wherein the composition contains about 150 to about 200 mM, optionally about 172 mM, sodium chloride.

9. The composition of any one of claims 1-8, wherein the composition contains about 2.5 to about 3.0 mM, optionally about 2.7 mM, potassium chloride.

10. The composition of any one of claims 1-9, wherein the composition contains about 5 to about 10 mM, optionally about 8 mM, disodium phosphate.

11. The composition of any one of claims 1-10, wherein the composition contains about 1.0 to about 2.0 mM, optionally about 1.5 mM, monopotassium phosphate.

12. The composition of any one of claims 1-11, wherein the composition contains about 0.5% to about 2% (w/v), optionally about 1% (w/v), sucrose.

13. The composition of any one of claims 1-12, wherein the composition contains about 0.01% to about 0.1% (w/v), optionally about 0.05% (w/v), poloxamer 188.

14. A pharmaceutical composition comprising
a recombinant adeno-associated virus (rAAV) vector,
about 171.81 mM sodium chloride,
about 2.68 mM potassium chloride,
about 8.10 mM disodium phosphate,
about 1.47 mM monopotassium phosphate,
about 1.40 mM magnesium chloride,
about 1.00% (w/v) sucrose, and
about 0.05% (w/v) poloxamer 188,
optionally wherein the composition comprises no more than about 0.1 mM calcium chloride and has a pH of about 7.1 to about 7.5.

15. A pharmaceutical composition comprising
a recombinant adeno-associated virus (rAAV) vector,
about 172 mM sodium chloride,
about 2.68 mM potassium chloride,
about 8.10 mM disodium phosphate,
about 1.47 mM monopotassium phosphate,
about 0.49 mM magnesium chloride,
about 1.00% (w/v) sucrose, and
about 0.05% (w/v) poloxamer 188,
optionally wherein the composition comprises no more than about 0.1 mM calcium chloride and has a pH of about 7.1 to about 7.5.

16. The composition of any one of claims 1-15, wherein the rAAV comprises a genome comprising an expression cassette for a therapeutic protein.

17. The composition of claim 16, wherein the therapeutic protein is a human factor VIII polypeptide.

18. A pharmaceutical composition comprising:
a recombinant adeno-associated virus (rAAV) vector, wherein the rAAV comprises a genome comprising an expression cassette for expressing a human factor VIII polypeptide,
about 171.81 mM sodium chloride,
about 2.68 mM potassium chloride,
about 8.10 mM disodium phosphate,
about 1.47 mM monopotassium phosphate,
about 1.40 mM magnesium chloride,
about 1.00% (w/v) sucrose, and
about 0.05% (w/v) poloxamer 188,
optionally wherein the composition comprises no more than about 0.1 mM calcium chloride and has a pH of about 7.1 to about 7.5.

19. A pharmaceutical composition comprising:
a recombinant adeno-associated virus (rAAV) vector, wherein the rAAV comprises a genome comprising an expression cassette for expressing a human factor VIII polypeptide
about 172 mM sodium chloride,
about 2.68 mM potassium chloride,
about 8.10 mM disodium phosphate,
about 1.47 mM monopotassium phosphate,
about 0.49 mM magnesium chloride,
about 1.00% (w/v) sucrose, and
about 0.05% (w/v) poloxamer 188,
optionally wherein the composition comprises no more than about 0.1 mM calcium chloride and has a pH of about 7.1 to about 7.5.

20. The composition of any one of claims 17-19, wherein the human factor VIII polypeptide comprises SEQ ID NO:1.

21. The composition of any one of claims 17-19, wherein the genome of the rAAV comprises SEQ ID NO:2 or nucleotides 131-5,024 of SEQ ID NO:2.

22. The composition of any one of claims 1-21, the composition contains the rAAV at about 1.0E+12 to about 1.0E+14 vector genomes (vg) per mL, optionally about 1.0E+13 to about 5.0E+13 vg per mL.

23. The composition of claim 22, wherein the composition contains about 1.0E+13 vg per mL.

24. The composition of any one of the preceding claims, wherein the rAAV comprises an AAV6 capsid protein, optionally inverted terminal repeats (ITRs) of AAV2.

25. A vial comprising 5-10 mL, optionally 6.4 mL, of the composition of any one of claims 1-24.

26. The vial of claim 25, wherein the vial is made of cyclo-olefin copolymer.

27. The vial of claim 25 or 26, wherein the vial has an in-place thermoplastic elastomer stopper.

28. A method of treating a patient in need of a therapeutic protein, comprising administering to the patient the composition of any one of claims 1-24.

29. A method of increasing the serum level of factor VIII in a human subject in need thereof, comprising administering intravenously to the human subject the pharmaceutical composition of any one of claims 17-24, or the entire content of the vial of any one of claims 25-27.

30. The method of claim 29, wherein the human subject has hemophilia A.

31. A method of treating hemophilia A in a human subject in need thereof, comprising administering intravenously to the human subject the pharmaceutical composition of any one of claims 17-24, or the entire content of the vial of any one of claims 25-27.

32. Use of the pharmaceutical composition of any one of claims 1-24 for the manufacture of a medicament for treating a human subject in a method of any one of claims 28-31.

33. The pharmaceutical composition of any one of claims 1-24 for use in treating a human subject in a method of any one of claims 28-31.
